(19) United States
(12) Patent Application Publication
(10) Pub. No.: US 2025/0242033 A1
(43) Pub. Date: Jul. 31, 2025

(54) Novel Stannic Protoporfin Compositions, Methods of Making, and Uses Thereof

(71) Applicant: Renibus Therapeutics, Inc., Southlake, TX (US)

(72) Inventors: Donald Jeffrey Keyser, Southlake, TX (US); Bhupinder Singh, Phoenix, AZ (US); Stacey Ruiz, Lantana, TX (US); Richard A. Zager, Seattle, WA (US); Dustin R. Cefalo, Logan, UT (US); David Owen Baumann, Logan, UT (US); Mark Acerson, Logan, UT (US); Raja Gabadage Waruna E. Jinadasa, Logan, UT (US)

(21) Appl. No.: 18/428,728

(22) Filed: Jan. 31, 2024

Related U.S. Application Data
(60) Provisional application No. 63/596,660, filed on Nov. 7, 2023.

(51) Int. Cl. A61K 47/54; A61K 9/00; A61K 33/26

(52) U.S. Cl. CPC A61K 47/546; A61K 9/0019; A61K 33/26

(57) ABSTRACT

Novel Stannic protoporfin compositions exhibiting high purity and other characteristics, including a novel solubility profile and visual indicators suggesting increase pharmacological activity, including enhanced antiviral activity. Also disclosed are novel processes for making Stannic protoporfin according to a process that requires fewer steps than known processes. Methods of treating patients before a scheduled insult such as surgy by administering a combination of stannic protoporfin and iron sucrose are provided. The present methods provide protection to photosensitive patients and those susceptible to post-operative complications.

### TECHNICAL FIELD

The present disclosure generally relates to novel stannic protoporfin compositions, methods of making novel stannic protoporfin compositions, and methods of therapeutic use of the novel stannic protoporfin compositions.

### BACKGROUND

Stannic protoporfin (Sn-protoporphyrin or SnPP) is known agent that undergoes proximal tubule uptake where it activates redox sensitive transcription factors, leading to the up-regulation of redox sensitive cytoprotective proteins. However, known methods of making SnPP includes a complicated process involving four distinct intermediate compounds and is known to have problems with stability and purity.

SnPP is disclosed in U.S. Pat. No. 10,639,321 B2, entitled “Compositions, kits, and methods to induce acquired cytoresistance using stress protein inducers.” The '321 patent discloses that SnPP in combination with iron sucrose (FeS) can be used to induce acquired cytoresistance. SnPP is also known to have some antiviral effect for particular kinds of viruses as disclosed by Neris et al., “Co-protoporphyrin IX and Sn-protoporphyrin IX inactivate Zika, Chikungunya and other arboviruses by targeting the viral envelope,” Sci Rep. 2018 Jun. 28; 8 (1): 9805. doi: 10.1038/s41598-018-27855-7. Neris et al. disclose that SnPP can be photosensitized and that non-photosensitized versions of SnPP may be used in photodynamic therapy for microorganism killing.

The synthesis of protoporphyrin from hemin has been explored by many researchers over the past several decades. The process typically involves the use of iron and an acid source. Iron is thought to facilitate reduction of the ferric iron in the ring system, which reduces the affinity of the metal ion to the ring, while the acid protonates the nitrogen atoms on the ring, which also reduces the affinity of the metal to the ring system. The existing processes of making SnPP have included unnecessary steps and have yielded compositions variable and sometimes undesirable impurities which may affect biological activity.

Photosensitivity issues upon administration of SnPP are well documented. See Land et al., “Photophysical studies of tin (IV)-protoporphyrin: Potential phototoxicity of a chemotherapeutic agent proposed for the prevention of neonatal jaundice,” Proc. Natl. Acad. Sci. USA, Vol. 85, pp. 5249-5253 (1988). Photosensitivity appears to have been a factor that previously led others to abandon SnPP for tin mesoporphyrin (SnMP) in the treatment of neonatal jaundice. Furthermore, the present inventors cited photosensitivity as an exclusion criteria in the original study design of the Phase II clinical trial, NCT04564833, entitled “Effect of RBT-1 on Preconditioning Response Biomarkers in Subjects Undergoing CABG and/or Cardiac Valve Surgery.” Specifically, this study excluded patients with “History of photosensitivity or active skin disease that, in the opinion of the Investigator, could be worsened by RBT-1.”

Accordingly, novel SnPP compositions having unique photodynamic and biologic properties are desired and that can be manufactured using convenient methods are needed.

### SUMMARY OF THE INVENTION

The present disclosure in one respect relates to stannic protoporfin compositions that includes a compound of Formula (I):

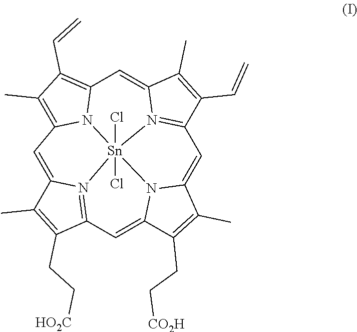

wherein the composition comprises a level of mesoporphyrin of Formula (III)

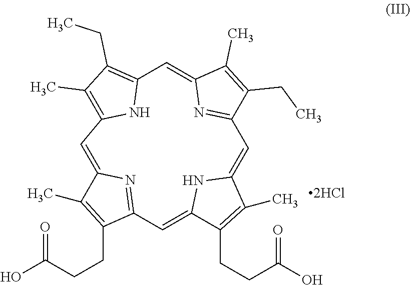 [SMILES: CCC1=C(C)c2cc3[nH]c(cc4nc(cc5[nH]c(cc1n2)c(C)c5CCC(=O)O)C(CCC(=O)O)=C4C)c(C)c3CC]

that is less than 1.0 wt. % and a total impurity level below 3 wt. %. The level of total impurities may be measured by gas chromatography and/or HPLC. The total level of impurities may be less than 1.5% by weight, or, for example, within the range of 0.01 to 1.5% by weight, 0.01 to 0.9% by weight, 0.1 to 0.9% by weight, 0.2 to 0.8% by weight, 0.3 to 0.7% by weight, 0.4 to 0.6% by weight, or about 0.5% by weight. The level of mesoporphyrin may be in the range of 0.01 to 0.9% by weight, 0.1 to 0.9% by weight, 0.2 to 0.8% by weight, 0.3 to 0.7% by weight, 0.4 to 0.6% by weight, or about 0.5% by weight. The total level of impurities may include mesoporphyrin and degradation products of tin protoporphyrin and intermediates of tin protoporphyrin either as the sole impurities, or among other impurities.

This composition may be preferably made from a process that includes two major steps. The process may include the following detailed process for making an protoporphyrin (IX) intermediate. For example, the method may include steps of (a) dissolving hemin in hot formic acid to form a first intermediate composition; (b) adding iron powder to the first intermediate composition to form a second intermediate composition; (c) adding a filtrate of the second intermediate composition to an aqueous solution of NH4OAc to precipitate a third intermediate composition; (d) dissolving the third intermediate composition in pyridine at an elevated temperature to form a fourth intermediate composition; (e) filtering the fourth intermediate composition to form a filtrate; and (f) precipitating a first intermediate compound according to Formula II:

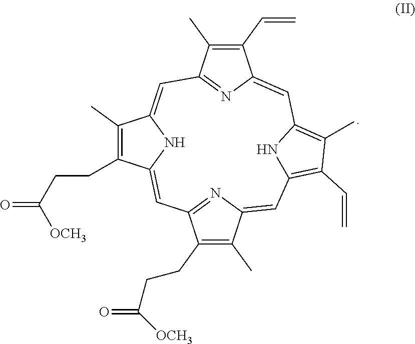 [SMILES: C=CC1=C(C)c2cc3[nH]c(cc4nc(cc5[nH]c(cc1n2)c(C)c5C=C)C(C)=C4CCC(=O)OC)c(CCC(=O)OC)c3C]

In another aspect, the method includes additions steps of adding the compound of Formula (II) to a mixture of stannous chloride, pyridine and glacial acetic acid to make a stannic protoporfin according to Formula I:

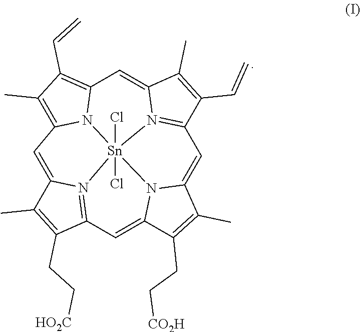

The addition of the compound of Formula (II) to the mixture of stannous chloride, pyridine and glacial acetic acid may be conducted in an inert environment.

In another aspect, the present invention includes compositions of stannic protoporfin having a total level of impurities of 1.5% or less, as measured by gas chromatography an iron sucrose bicarb. In another aspect, the SnPP having a total level of impurities of 1.5% or less is combined with iron sucrose and administered to a patient scheduled to undergo cardiothoracic surgery.

In another aspect, the present disclosure relates to a method for demetallation of a vinyl group-containing metallomacrocycle, comprising: reacting the vinyl group-containing metallomacrocycle with a transition metal having an oxidation number of 0 in a mixture comprising an acid and an olefin. The vinyl group-containing metallomacrocycle may be selected from the group consisting of a metalloporphyrin, a metallochlorin, a metallocorrole, and any combination thereof. The addition of the vinyl group-containing metallomacrocycle to the acid may form a first mixture, and addition of the olefin to the first mixture may form a second mixture. In one aspect, the transition metal may be added to the second mixture.

In one respect, the present disclosure relates to a composition, comprising: a vinyl group-containing metallomacrocycle, an acid, an olefin, and a transition metal having an oxidation number of 0. This composition may be used in the production of stannic protoporfin to remove the iron from a hemin source material.

The present disclosure also relates to a method of reducing post operative complications of a human patient from injury based on a scheduled or anticipated surgical operation, the method comprising administering to the human patient a composition comprising a therapeutically effective amount of (i) an iron compound; and (ii) stannic protoporfin in a dose of 20-80 mg before the surgical operation, wherein the human patient is susceptible to photodermatoses. The stannic protoporfin may be as described in the paragraphs above, and/or manufactured according to one or more of the processes described above.

In aspects where the patient is photosensitive, the method may involve applying sunscreen to the patient's skin within six days after the scheduled or anticipated surgical operation. The sunscreen may be applied to the patient's skin before any expected sun exposure within six days after the scheduled or anticipated surgical operation. In one aspect, the human patient may be instructed to or may desire to reduce or eliminate sun exposure between the administration of the therapeutically effective composition and the scheduled or anticipated surgical operation. The human patient may apply sunscreen when sunlight when exposure is expected for at least 6 days after the scheduled or anticipated surgical operation. In one aspect, sunscreen is administered when sun exposure is expected after the therapeutically effective composition is administered. The sunscreen may have a sun protection factor (SPF) of 50+.

In another aspect, the present invention relates to a method of reducing post operative complications of a human patient from injury based on a scheduled or anticipated insult to an organ of the human patient, the method comprising: (a) administering to the patient a therapeutically effective composition comprising (i) an iron compound; and (ii) stannic protoporfin, and (b) applying sunscreen to the human patient's skin around the time of administering to the human patient the therapeutically effective composition or within six days after the scheduled or anticipated surgical operation. The sunscreen may be applied to the patient's skin before any expected sun exposure within six days after the scheduled or anticipated surgical operation. In one aspect, the human patient may be instructed to or may desire to reduce or eliminate sun exposure between the administration of the therapeutically effective composition and the scheduled or anticipated surgical operation. The human patient may apply sunscreen when sunlight when exposure is expected for at least 6 days after the scheduled or anticipated surgical operation. In one aspect, sunscreen is administered when sun exposure is expected after the therapeutically effective composition is administered. The sunscreen may have a sun protection factor (SPF) of 50+.

In another aspect, the present invention relates to a method of reducing hospital readmission for cardiopulmonary purposes of a human patient after a surgical operation by at least 60% comprising administering to the patient a therapeutically effective composition comprising an amount of (i) an iron compound; and (ii) stannic protoporfin before the surgical operation. The method may result in a rate of readmission for cardiopulmonary purposes that is reduced by at least 70%, or more specifically 72%. The human patient may be selected from a patient that has an elevated risk for hospital readmission for cardiopulmonary purposes after receiving and CABG and/or heart valve surgery.

In another aspect, the present invention relates to a method of reducing post operative complications of a human patient from injury based on a scheduled or anticipated surgical operation comprising administering to the patient a therapeutically effective composition comprising an amount of (i) an iron compound; and (ii) stannic protoporfin before the surgical operation, wherein the post operative complications include: (a) greater than three days in the intensive care unit, (b) greater than 24 hours on a ventilator, (c) readmission for cardiopulmonary surgery, (d) need for a blood transfusion, (e) new onset post-operative atrial fibrillation (POAF) during hospitalization, or a combination of two or more of (a)-(e). The human patient may be selected from a patient that has an elevated risk post-operative complications including one or more of the above-listed complications after receiving and CABG and/or heart valve surgery.

In any of the above embodiments, the stannic protoporfin may be administered in a dose of 20-80 mg, more preferably at a dose of 30-70 mg, or a dose of 45 mg. The iron sucrose may be administered at a dose of 190-290 mg, or preferably at a dose of 240 mg. In one embodiment, the stannic protoporfin is administered at a dose of 45 mg and iron compound is iron sucrose is administered at a dose of 240 mg.

In any of the above embodiments, the iron compound may be present in an aqueous pharmaceutical composition comprising: iron sucrose; bicarbonate; and a pharmaceutically acceptable aqueous carrier. The stannic protoporfin may be present in an aqueous pharmaceutical composition comprising: a total level of impurities of 1.5% or less, as measured by gas chromatography. In one aspect, the preparation of the therapeutically effective composition for administration may comprise combining in an intravenous bag: (a) an aqueous iron pharmaceutical composition comprising: iron sucrose; bicarbonate; and a pharmaceutically acceptable aqueous carrier; and (b) a stannic protoporfin composition having a total level of impurities of 1.5% or less, as measured by gas chromatography.

In one aspect, the therapeutically effective amount of stannic protoporfin may be administered at a dose of 0.5-0.8 mg/kg, more preferably 0.6-0.7 mg/kg, and particularly at 0.63 mg/kg. In another aspect, the therapeutically effective amount of iron sucrose may be 2-6 times the amount by weight of stannic protoporfin, or more preferably 3-4 times the amount of stannic protoporfin.

In one aspect, the scheduled or anticipated surgical operation is surgery to an organ. The organ may be heart, kidney, liver or lung. In one aspect, the iron compound and the stannic protoporfin are administered at least 24 hours before the scheduled or anticipated surgical operation to the organ occurs. In one aspect, it is desirable that the iron compound and the stannic protoporfin be administered no more than 48 hrs before the scheduled or anticipated surgical operation to the organ occurs.

In one aspect, the scheduled or anticipated surgical operation may be a surgery comprising coronary artery bypass graft (CABG), cardiac valve, or combined CABG/valve surgery on cardiopulmonary bypass (CPB). In another aspect, the scheduled or anticipated surgical operation is organ transplant surgery. In the case of organ transplantation, the method may reduce or eliminate the need for a blood product to be administered after the scheduled or anticipated surgical operation. This may include whole blood products from a donor.

The foregoing has outlined rather broadly various features and technical advantages of the present disclosure in order that the detailed description that follows may be better understood. Additional features and advantages of the disclosure will be described hereinafter that form the subject of the claims of this application.

### BRIEF DESCRIPTION OF THE DRAWINGS

FIG. 1A shows the first step in the production of SnPP according to a comparative example;

FIG. 1B shows the second step in the production of SnPP according to the comparative example;

FIG. 1C shows the third step in the production of SnPP according to the comparative example;

FIG. 1D shows the fourth step in the production of SnPP according to the comparative example;

FIG. 2A shows the first step in the production of SnPP according to an example according to an embodiment of the invention;

FIG. 2B shows the second step in the production of SnPP according to an example according to an embodiment of the invention;

FIG. 3 shows a comparison of the impurity profiles present in the product of Example 1 compared to the Comparative Example;

FIG. 4 shows % cell viability after exposure to SnPP for Example 1 compared to the Comparative Example;

FIG. 5 shows a flowchart illustrating the design of the study assessing RBT-1 to reduce post-operative complications in patients undergoing cardiothoracic surgery;

FIG. 6 shows a statistically significant increase in cytoprotective response biomarkers HO-1, Ferritin, and IL-10 with RBT-1;

FIG. 7 on the left graph shows the percentage of subjects in each group that were on a ventilator for more than 24 hours post cardiac surgery and on the right graph shows the percentage of subjects that where in the ICU for greater than 3 days;

FIG. 8 shows the average time spent for each group on a ventilator, in the ICU, and in the hospital following cardiac surgery;

FIG. 9 shows the improvement in the mean composition of hospitalization in patients treated with RBT-1;

FIG. 10 shows the median Troponin I levels through the first day post-operation;

FIG. 11 shows the improvement of postoperative outcomes in patients treated with RBT-1 undergoing cardiac surgery;

FIG. 12 shows the improvement of postoperative outcomes in patients treated with RBT-1 undergoing cardiac surgery;

FIG. 13 shows a flow diagram of patient populations according to Example 5.

FIG. 14 shows the mean composite of the maximum pre-operative change from baseline in HO-1, IL-10, and ferritin from Example 5.

FIG. 15 shows assessment of hospitalization course by time on ventilator and length of stay (LOS) in the ICU and hospital according to Example 5.

FIG. 16 shows the primary outcome-preconditioning response in accordance with Example 6.

FIG. 17 shows the ventilator, ICU and hospitalization course in accordance with Example 6.

FIG. 18 shows post-operative troponin I in accordance with Example 6.

FIG. 19 is a table showing the primary outcome for interim population in accordance with Example 6.

FIG. 20 is a table showing baseline characteristics for the safety population in accordance with Example 6.

FIG. 21 is a table showing baseline characteristics for the modified intent-to-treat population in accordance with Example 6.

FIG. 22 is a table showing other prespecified outcomes for the modified intent-to-treat population in accordance with Example 6.

FIG. 23 is a table showing hierarchical composite outcome (win ratio) in accordance with Example 6.

FIG. 24 shows a comparison of ventilator, ICU times, and cardiopulmonary readmissions for Phase 2 patients undergoing CABG and/or valve surgery on cardiopulmonary bypass.

FIG. 25 shows the design for a Phase III study of RBT-1.

### DETAILED DESCRIPTION

The present disclosure relates to novel compositions of stannic protoporfin made according to a novel process that reduces the number of steps required relative to processes used to produce known stannic protoporfin compositions, and that exhibit an improved impurity profile and pharmacological properties relative to known Stannic protoporfin compositions.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

“RBT-1” is synonymous with comprises a combination of stannic protoporfin and iron sucrose that has been used to generate data disclosed in this application.

SnPP composition according to an embodiment of the invention includes a compound of Formula (I):

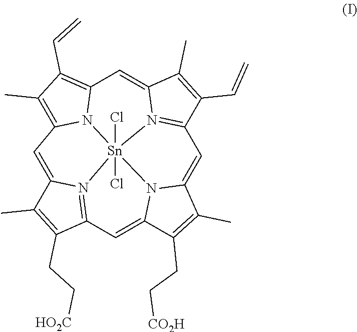

having a total level of impurities of 3% by weight or less, as measured by gas chromatography or high performance liquid chromatography (HPLC). The total level of impurities is preferably less than 1.5% by weight. In an embodiment, the total level of impurities may be 1.5% or less, or preferably 1% or less. In other embodiments the level of impurities is within a range of 0.01 to 1%, 0.1 to 1%, 0.2 to 1%, 0.3 to 1%, 0.4 to 1%, 0.5 to 1%, 0.6 to 1%, 0.7 to 1%. In another aspect, the total level of impurities is between 0.1 to 0.9% by weight, 0.2 to 0.8% by weight, 0.3 to 0.7% by weight, 0.4 to 0.6% by weight, or about 0.5% by weight.

This composition may be preferably made from a process that includes two major steps. The process may include the following detailed demetallation process for making an protoporphyrin (IX) intermediate. For example, the method may include steps of (a) dissolving vinyl group-containing metallomacrocycle, e.g., hemin, in an acid, e.g., formic acid, under heat to form a first intermediate composition; (b) adding a transition metal having an oxidation number of 0, e.g., iron powder, to the first intermediate composition to form a second intermediate composition; (c) adding a filtrate of the second intermediate composition to an aqueous solution of NH₄OAc to precipitate a third intermediate composition; (d) dissolving the third intermediate composition in pyridine at an elevated temperature to form a fourth intermediate composition; (e) filtering the fourth intermediate composition to form a filtrate; and (f) precipitating a first intermediate compound, protoporphyrin (IX), according to Formula II:

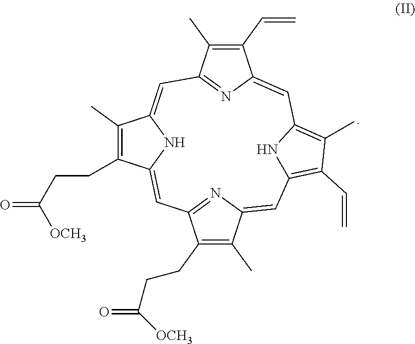 [SMILES: C=CC1=C(C)c2cc3[nH]c(cc4nc(cc5[nH]c(cc1n2)c(C)c5C=C)C(C)=C4CCC(=O)OC)c(CCC(=O)OC)c3C]

The above reaction is highly sensitive to the presence of oxygen, which may cause the reaction to stall and not go to completion. In some respects, it is therefore desirable to provide a steady flow of inert gas during the reaction. In addition, the level of oxygen in the reactor may be monitored and controlled to be below 1% using an oxygen sensor. The above reaction produces mesoporphyrin as a by-product along with decomposition products of the SnPP. These impurities may be controlled by limiting the amount of time the reaction is heated and temperature after the final addition of iron powder. For example, rapid cooling of the reaction limits unwanted impurities from forming.

As an alternative to the above-noted demetallation process, the vinyl group-containing metallomacrocycle, e.g., hemin, may be reacted with a transition metal having an oxidation number of 0, e.g., iron powder, in the mixture comprising an acid, e.g., formic acid, and further including an olefin, e.g., cyclohexene. This alternative method has been shown to further reduce the levels of mesoporphyrin, according to Formula (III):

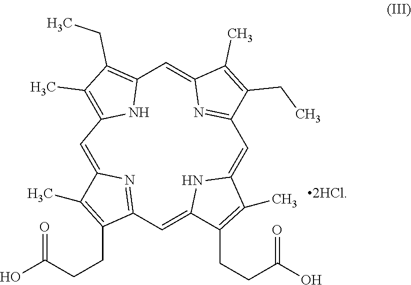 [SMILES: CCC1=C(C)c2cc3[nH]c(cc4nc(cc5[nH]c(cc1n2)c(C)c5CCC(=O)O)C(CCC(=O)O)=C4C)c(C)c3CC]

In one aspect, the level of mesoporphyrin impurity in the intermediate protoporphyrin (IX) composition that is made using one or more of the above-noted processes, or variations described above, may be less than 1.5%. More preferably, the level of mesoporphyrin impurity in the intermediate protoporphyrin (IX) composition is less than 1%. In one aspect the level of mesoporphyrin impurity in the intermediate composition is about 0.5% as measured by HPLC. In an embodiment, the level of mesoporphyrin impurity in the intermediate protoporphyrin (IX) composition is 1.5% or less, or preferably 1% or less. In other embodiments the level of mesoporphyrin impurity in the intermediate protoporphyrin (IX) composition is within a range of 0.01 to 1%, 0.1 to 1%, 0.2 to 1%, 0.3 to 1%, 0.4 to 1%, 0.5 to 1%, 0.6 to 1%, 0.7 to 1%. In another aspect, the level of mesoporphyrin impurity in the intermediate protoporphyrin (IX) composition is between 0.01 to 0.9% by weight, 0.1 to 0.9% by weight, 0.2 to 0.8% by weight, 0.3 to 0.7% by weight, 0.4 to 0.6% by weight, or about 0.5% by weight, or any intermediate range of the listed endpoints.

In another aspect, the method includes producing a stannic protoporfin and includes additional steps of adding the compound of Formula (II) from any of the above processes to a mixture of stannous chloride, pyridine and glacial acetic acid to make a Stannic protoporfin according to Formula I:

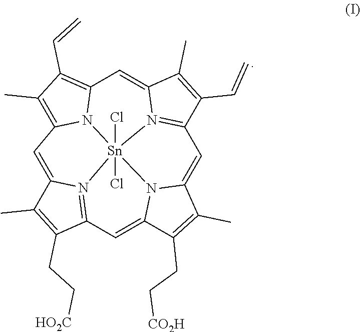

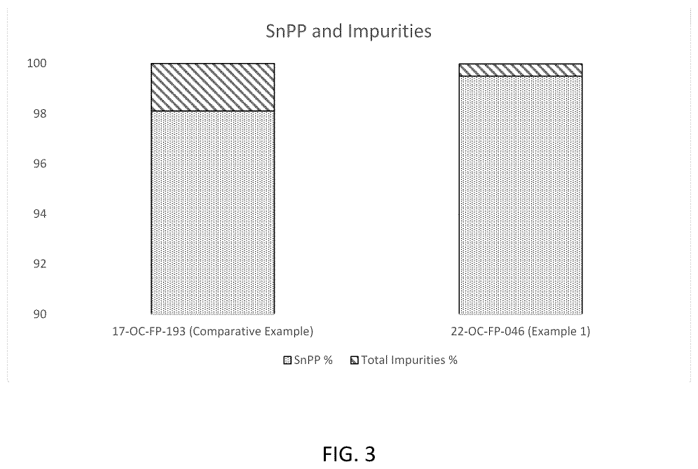

In one aspect, the stannic protoporfin composition has a total level of impurities of 3% or less, as measured by gas chromatography. In an embodiment, the level of impurities is 1.5% or less, or preferably 1% or less. In other embodiments the level of impurities is within a range of 0.01 to 1%, 0.1 to 1%, 0.2 to 1%, 0.3 to 1%, 0.4 to 1%, 0.5 to 1%, 0.6 to 1%, 0.7 to 1%. In another aspect, the total level of impurities is between 0.01 to 0.9% by weight, 0.1 to 0.9% by weight, 0.2 to 0.8% by weight, 0.3 to 0.7% by weight, 0.4 to 0.6% by weight, or about 0.5% by weight. The chromatography method includes, for example, the chromatography method to produce the experimental results in Table I and FIG. 3. Other methods of determination of impurity levels such as HPLC may be used as well.

In another aspect, the final stannic protoporfin composition includes a total level of impurities as noted above and further a level of mesoporphyrin that is less than 1.5% by weight, less than 1% by weight, less than 0.5% by weight. In other embodiments, the level of mesoporphyrin impurity is within a range of 0.01 to 1%, 0.1 to 1%, 0.2 to 1%, 0.3 to 1%, 0.4 to 1%, 0.5 to 1%, 0.6 to 1%, 0.7 to 1%. In another aspect, the level of mesoporphyrin impurity is between 0.01 to 0.9%, 0.1 to 0.9% by weight, 0.2 to 0.8% by weight, 0.3 to 0.7% by weight, 0.4 to 0.6% by weight, or about 0.5% by weight.

The present inventors have determined that the SnPP compositions according to aspects of the invention resulted in an improved biological activity. In some cases, the improved biological activity allowed reduction in the amount of SnPP required for certain therapeutic applications. For example, it was unexpectedly found that the SnPP compositions present herein exhibited an equivalent therapeutic efficacy could be obtained when SnPP is combined with iron sucrose using half the amount of SnPP previously thought needed, 45 mg versus 90 mg. This advantageously reduced side effects, including photosensitivity, which allowed treatment of patients who are photosensitive.

The processes disclosed herein advantageously eliminate or substantially eliminate unwanted byproducts, such as mesoporphyrin if hemin is used as a starting material. In contrast, mesoporphyrin is typically present in amounts ranging from about 5 wt. % to about 20 wt. % of the total reaction product produced according to prior art processes directed to producing protoporphyrin from hemin.

The processes disclosed herein also significantly improve the desired reaction product yields as compared to the yields obtained with prior art processes. A variety of process parameters were extensively studied to determine how each parameter could be modified to achieve a substantial improvement in the overall process.

For example, reaction temperatures were examined and it was determined that significant improvements in the overall quality of the crude material could be obtained by lowering the reaction temperature. However, by lowering the reaction temperature, the amount of time needed to force the reactions to completion was significantly increased. The longer reaction times also led to increased reduction of the vinyl groups present on the metallomacrocycle, which is an unwanted reaction that produces, for example, mesoporphyrin if hemin is used as a starting material.

Additionally, the inventors studied the effect of oxygen being present during the reactions on the quality of the final crude product. It was observed that the presence of oxygen led to significant increases in unidentified impurities/byproducts formed during the process. The inventors determined that carrying out the reactions in a reduced oxygen environment produced significantly cleaner products (e.g., reduced byproduct formation) and increased product yields.

In one optional aspect, the inventors added an olefin to the process in an attempt to further limit reduction of the vinyl groups present on the metallomacrocycle. The inventors discovered that the presence of an olefin had two effects. First, the amount of unwanted byproduct (e.g., mesoporphyrin) formed in the reaction was significantly reduced (e.g., less than 3%) or eliminated entirely in certain embodiments. Second, the presence of the olefin significantly reduced the rate of the metal (e.g., iron) removal from the metallomacrocycle (e.g., hemin) during the process. The reduced reactivity led to significantly longer reaction times and led to an increased amount of vinyl group reduction during the extended reaction times.

The inventors discovered that the addition of a phase transfer catalyst could overcome the slower metal removal rates in the reactions with added olefins. The addition of the phase transfer catalyst significantly increased the overall reactivity of the process, shortening the length of the process from about 24-48 hours to about 4-5 hours.

The foregoing discoveries led to the invention of processes that produce a high-purity crude product (i.e., >97% by HPLC) with significantly improved yields (i.e., 90% or more). The processes also limit reduction of the vinyl groups present on the metallomacrocycle. For example, when the processes of the present disclosure were carried out with hemin as a starting material, the amount of mesoporphyrin produced was either reduced to zero or at least below 2% (as compared to 5-20% using the prior art processes).

When carrying out the processes disclosed in the present application, the order of addition of the various reactants is not particularly limited and all orders of addition are intended to be covered by the present disclosure. In certain illustrative, non-limiting embodiments, the vinyl group containing metallomacrocycle may be added to the acid to form a first mixture. The olefin may be added to the first mixture to form a second mixture. A phase transfer catalyst may optionally be added to the first and/or second mixture.

In additional illustrative embodiments, the vinyl group-containing metallomacrocycle may be added to the olefin to form a first mixture and the acid may be added to the first mixture to form a second mixture. Again, a phase transfer catalyst may optionally be added to the first and/or second mixture.

A transition metal may be added to any mixture, such as the second mixture, and, in some embodiments, a mixture comprising the vinyl group containing metallomacrocycle, the acid, the olefin, the transition metal, and optionally the phase transfer catalyst may be added to a solution comprising a buffer.

Again, the present disclosure covers any order of addition of the various reactants, such as mixing the acid with the olefin and adding the resulting mixture to the vinyl group-containing metallomacrocycle, followed by addition of the transition metal and optionally the phase transfer catalyst; mixing the acid with the vinyl group-containing metallomacrocycle and a phase transfer catalyst and adding the resulting mixture to the olefin, followed by addition of the transition metal; mixing the olefin with the vinyl group containing metallomacrocycle and a phase transfer catalyst and adding the resulting mixture to the acid, followed by addition of the transition metal; etc.

Regarding any of the above processes, the reactants may be added all at once or they may be added in various portions. For example, when adding the transition metal, it may be added to a mixture all at once or it may be divided into various portions, such as 2, 3, 4, 5, 6, 7, 8, 9, 10, or more, portions and each portion may be added separately to the mixture. For example, a first portion could be added to a mixture and then, for example, every five minutes, another portion could be added to the mixture until all portions have been added.

The methods disclosed herein may optionally comprise conducting all or some of the reactions in a solvent. For example, a solvent may comprise a mixture of the metallomacrocycle with the acid, and the olefin in the case that an olefin is used, and optionally the transition metal and/or the phase transfer catalyst. In certain embodiments, the acid and the olefin may account for about 80 wt. % to about 100 wt. % of the solvent, such as from about 85 wt. % to about 100 wt. %, about 90 wt. % to about 100 wt. %, or about 95 wt. % to about 100 wt. %. In other aspect where an olefin is not used, the acid may account for about 80 wt. % to about 100 wt. % of the solvent, such as from about 85 wt. % to about 100 wt. %, about 90 wt. % to about 100 wt. %, or about 95 wt. % to about 100 wt. %.

Additional examples of solvents include, but are not limited to, dimethylformamide, dimethylsulfoxide, N-methylpyrrolidine, N, Ndimethylacetamide, and any combination thereof.

The methods disclosed herein optionally include heating any mixture disclosed herein, such as the first mixture, the second mixture, and/or a mixture comprising all of or any combination of the vinyl group-containing metallomacrocycle, the transition metal, the acid and any other optional components associated with certain embodiments, such as the olefin, the buffer, and the phase transfer catalyst.

Any mixture disclosed herein may be heated to a temperature of about 40° C. to about 140° C., such as from about 60° C. to about 140° C., about 80° C. to about 140° C., about 100° C. to about 140° C., about 120° C. to about 140° C., about 40° C. to about 120° C., about 40° C. to about 100° C., about 40° C. to about 80° C., about 40° C. to about 60° C., about 60° C. to about 120° C., or about 80° C. to about 100° C. In some embodiments, a mixture is heated to any of the foregoing temperatures before the transition metal is added.

Any mixture disclosed in the present application may also be cooled. For example, a mixture of the vinyl group-containing metallomacrocycle, the acid, the olefin if one is used, and optionally a phase transfer catalyst may be cooled before and/or after adding the transition metal.

Additionally, any reactions disclosed herein may be conducted at room temperature (e.g., about 20° C. to about 23° C.). In some embodiments, a mixture may be at room temperature while adding one or more reactants and after and/or during the addition, the mixture may be cooled or heated. Alternatively, a mixture may be below room temperature while adding one or more reactants and after and/or during the addition, the mixture may heated or allowed to rise to room temperature. As an additional illustrative example, a mixture may be at a temperature above room temperature while adding one or more reactants and after and/or during the addition, the mixture may cooled or allowed to drop to room temperature.

When carrying out the methods disclosed in the present application, various amounts of the components may be used. For example, in embodiments that use an olefin, the olefin may be added at a molar ratio of olefin to vinyl group-containing metallomacrocycle of about 1:1 to about 50:1, such as from about 1:1 to about 40:1, about 1:1 to about 30:1, about 1:1 to about 20:1, about 1:1 to about 10:1, about 1:1 to about 5:1, about 1:1 to about 4:1, about 1:1 to about 3:1, or about 1:1 to about 2:1.

As an additional example, the transition metal may be added at a molar ratio of transition metal to vinyl group-containing metallomacrocycle of about 0.5:1 to about 10:1, about 0.5:1 to about 8:1, about 0.5:1 to about 6:1, about 0.5:1 to about 4:1, about 0.5:1 to about 2:1, or about 0.5:1 to about 1:1.

Further, the acid may be added at a molar ratio of acid to vinyl group-containing metallomacrocycle of about 1:1 to about 100:1, such as from about 1:1 to about 90:1, about 1:1 to about 80:1, about 1:1 to about 70:1, about 1:1 to about 60:1, about 1:1 to about 50:1, about 1:1 to about 40:1, about 1:1 to about 30:1, about 1:1 to about 20:1, about 1:1 to about 10:1, about 1:1 to about 5:1, about 1:1 to about 4:1, about 1:1 to about 3:1, or about 1:1 to about 2:1.

If a phase transfer catalyst is added to any mixture disclosed herein, the catalyst loading may be from about 0.1% to about 10%, such as from about 0.1% to about 8%, about 0.1% to about 6%, about 0.1% to about 4%, about 0.1% to about 2%, about 0.1% to about 1%, or about 0.1% to about 0.5% of the weight of the mixture.

Any reaction disclosed herein may optionally be carried out in a reduced-oxygen environment. Further, any mixture disclosed herein may be present in a reduced-oxygen environment. A reduced-oxygen environment may be achieved by, for example, sparging the environment with an inert gas (e.g., He, Ne, Ar, Kr, Xe, or Rn). In some embodiments, the environment may be sparged with nitrogen and/or carbon dioxide. A reduced-oxygen environment may include, for example, less than about 10% by volume of oxygen, such as from about 0% to about 10%, about 0% to about 8%, about 0% to about 5%, about 0% to about 2%, about 0.1% to about 2%, about 0.1% to about 5%, about 0.5% to about 3%, or about 1% to about 2% oxygen by volume.

By way of an illustrative example, the methods disclosed in the present application may be used to prepare protoporphyrin IX. For example, a method may include providing a mixture comprising hemin, an acid, and optionally an olefin. The method may also include a step of adding a transition metal having an oxidation number of 0 to the mixture. The hemin, acid, and optional olefin and/or transition metal may be added to the mixture simultaneously or in any order, such as hemin, acid, olefin, transition metal; acid, hemin, olefin, transition metal; hemin, olefin, acid, transition metal; acid, olefin, hemin, transition metal; etc. Any phase transfer catalyst disclosed herein may also be added before, after, and/or with the olefin, acid, transition metal, and/or hemin. Also, as disclosed herein, heating and/or cooling may be used when adding any of the reactants.

After allowing the hemin, acid, and optional olefin and/or transition metal to react, the method may further include precipitating protoporphyrin IX from the mixture. Precipitation may be carried out by, for example, filtering the reaction mixture and pouring the filtered reaction mixture into a stirring solution of a buffer.

After allowing the hemin, acid, and optional olefin and/or transition metal to react (or after quenching, filtering, precipitating, and/or drying), the mixture (or reaction product) comprises less than about 20 wt. % of mesoporphyrin, such as from about 0 wt. % to about 15 wt. %, about 0 wt. % to about 10 wt. %, about 0 wt. % to about 5 wt. %, about 0 wt. % to about 2 wt. %, or about 0 wt. %, about 1 wt. %, about 2 wt. %, or about 3 wt. % of mesoporphyrin.

Also, after allowing the hemin, acid, olefin, and transition metal to react (or after quenching, filtering, precipitating, and/or drying), the mixture (or reaction product) comprises greater than about 50 wt. % of protoporphyrin IX, such as from about 50 wt. % to about 99 wt. %, about 60 wt. % to about 99 wt. %, about 70 wt. % to about 99 wt. %, about 80 wt. % to about 99 wt. %, or about 90 wt. % to about 99 wt. % of protoporphyrin IX.

The methods of the present disclosure may be carried out with a metallomacrocycle. The metallomacrocycle may include, for example, a metalloporphyrin, a metallochlorin, a metallocorrole, and any combination thereof. In certain embodiments, the vinyl group-containing metallomacrocycle is hemin.

The vinyl group-containing metallomacrocycle may comprise a variety of metals. For example, the metallomacrocycle may comprise a metal selected from the group consisting of iron, manganese, titanium, zinc, aluminum, magnesium, chromium, scandium, vanadium, cobalt, nickel, copper, and any combination thereof. In some embodiments, the metal comprises an oxidation state of +3.

The acid used in the methods disclosed herein is not particularly limited and may include, for example, an organic acid and/or an inorganic acid. Illustrative, non-limiting examples of acids include acetic acid, formic acid, propionic acid, butyric acid, trichloroacetic acid, trifluoroacetic acid, sulfuric acid, phosphoric acid, nitric acid, hydrochloric acid, hydrofluoric acid, and any combination thereof.

The olefin used in the methods disclosed herein is not particularly limited and may include, for example, a linear, branched, and/or cyclic C₂ olefin to a C₅₀ olefin, such as a C₂ olefin to a C₄₀ olefin, a C₂ olefin to a C₃₀ olefin, a C₂ olefin to a C₂₀ olefin, a C₂ olefin to a C₁₅ olefin, a C₂ olefin to a C₁₀ olefin, a C₂ olefin to a C₅ olefin, a C₅ olefin to a C₃₀ olefin, a C₁₀ olefin to a C₂₀ olefin, a C₂₀ olefin to a C₃₀ olefin, or a C₂₅ olefin to a C₅₀ olefin.

The olefin may comprise, for example, an ether or ester bond linkage. As used herein, the term “olefin” is intended to include a polyolefin. In accordance with certain embodiments, any olefin may be used in the methods of the present disclosure so long as it has a boiling point equal to or higher than the reaction temperature. In some embodiments, the olefin is a naturally derived olefin, such as pinene or limonene.

Illustrative, non-limiting examples of olefins may be selected from the group consisting of ethene, propene, cyclopropene, butene, cyclobutene, pentene, cyclopentene, hexene, cyclohexene, heptene, cycloheptene, limonene, pinene, and any combination thereof.

The transition metal used in accordance with the methods disclosed herein comprises an oxidation number of 0. The transition metal may be selected from, for example, iron, zinc, manganese, magnesium, and any combination thereof. In certain embodiments, a powder comprises the transition metal.

A variety of phase transfer catalysts may be used in accordance with the methods disclosed in the present application. For example, the phase transfer catalyst may comprise the formula [NR4]+X-, wherein each R is independently selected from an alkyl group, an alkenyl group, or an aryl group, and X is a halide. Illustrative, non-limiting examples of phase transfer catalysts include benzyl triethylammonium chloride, cetyl trimethylammonium chloride, octadecyl trimethylammonium chloride, and any combination thereof. In some embodiments, the phase transfer catalyst comprises a nonionic surfactant.

The buffer used in accordance with certain methods disclosed herein is not particularly limited. Illustrative examples of the buffer include ammonium acetate, sodium acetate, potassium acetate, ammonium propionate, ammonium hydroxide, sodium hydroxide, and any combination thereof. Any base (organic or inorganic) could be used to neutralize the acid disclosed herein. For example, ammonium acetate can act as a buffer to neutralize formic acid but other buffer combinations could be used, such as a salt of an acid that is weaker than formic acid (or weaker than whatever acid is chosen for the reaction).

The buffer solution may be useful for quenching any reaction disclosed in the present application. Quenching may take place, if at all, immediately after a reaction is complete, within about 5 minutes after a reaction is complete, within about 15 minutes after a reaction is complete, within about 30 minutes after a reaction is complete, with about 1 hour after a reaction is complete, within about 6 hours after a reaction is complete, within about 12 hours after a reaction is complete, within about 18 hours after a reaction is complete, within about 24 hours after a reaction is complete, or more than about 24 hours after a reaction is complete, such as about 36 hours, about 48 hours, etc., after a reaction is complete.

In addition to methods, the present disclosure also provides various compositions. A composition of the present disclosure may comprise one or more of the reactants/components disclosed herein. For example, a composition may comprise, consist of, or consist essentially of any vinyl group-containing metallomacrocycle disclosed herein, any acid disclosed herein, any olefin disclosed herein, any transition metal disclosed herein, and optionally any phase transfer catalyst disclosed herein.

The iron sucrose used in RBT-1 is an iron sucrose composition that can be readily combined with tin protoporphyrin (SnPP) to form a stable solution that can be administered to a patient, as described in U.S. Pat. No. 11,292,813 filed Apr. 5, 2022, entitled “NOVEL IRON COMPOSITIONS AND METHODS OF MAKING AND USING THE SAME,” the subject matter of which is incorporated herein by reference. The composition, also known as RBT-3, is an aqueous iron pharmaceutical composition comprising: iron sucrose; bicarbonate; and a pharmaceutically acceptable aqueous carrier, wherein the iron sucrose is present in pharmaceutically effective amount for providing a protective effect to a patient's organs the iron being present in both iron (II) and iron (III) form, the pharmaceutical composition has a pH greater than 9, a concentration of iron (II) of 0.05% w/v to 0.41% w/v, and the iron sucrose has a MW according to GPC of between 33,000 and 38,000 Daltons. RBT-3 (iron sucrose) is a low molecular weight iron nanoparticle that has the potential to rapidly restore iron levels and improve blood product utilization in cardiac surgery and/or ER patients. RBT-3 has also demonstrated the potential to mitigate cisplatin induced nephrotoxicity in preclinical models. We are currently exploring opportunities to further the clinical development of RBT-3 in these potential indications.

The iron sucrose composition can be prepared, for example, by dissolving enough iron sucrose complex in water (ca 3.5 L) to give a 12 mg/mL (expressed as iron) solution when diluted to 6.0 L. The amount of iron sucrose needed was calculated for the final volume of liquid, 6100 mL (6.1 L) so that the final concentration is 12 mg/ml. This requires 73.2 g of iron. The use potency of iron sucrose is 0.0550. Thus, 73.2 g/0.0550 or 1331 g±1 g of iron sucrose is needed. Iron sucrose, 1331 g±1 g, was weighed directly into a 6.0 L Erlenmeyer flask. Approximately 3-3.5 L of water is added to the Erlenmeyer flask, and the contents of the flask are stirred.

Sodium bicarbonate is added in an amount such that the final sodium bicarbonate concentration is 10 mg/ml when diluted to 6.0 L. Sodium bicarbonate, 109.8±0.1 g, is weighed and added to the 6.0 L flask. Sodium chloride is added in an amount such that the final sodium chloride concentration is 9.0 mg/ml upon dilution. Sodium chloride, 54.9±0.1 g, is weighed and added to the 6.0 L flask. The suspension is stirred for 30-120 minutes to give a black opaque solution. The pH of the solution is monitored with a pH meter while 1 M sodium hydroxide is added in small portions until pH 10.30 is reached and remains stable. Sodium hydroxide, 40.0±0.1 g, was added to a 1.0 L Erlenmeyer flask. 1.0±0.1 L of water is added to the 1.0 L Erlenmeyer flask and stirred until all of the sodium hydroxide dissolved. A pH probe is affixed to monitor the pH of the 6.0 L Erlenmeyer flask and the sodium hydroxide is added in <100 mL portions until the pH=10.3±0.1. The solution is then stirred for 10 minutes. The pH is checked again after 10 minutes and if necessary adjusted to within pH=10.3±0.1.

The solution is then transferred to a volumetrically accurate flask and diluted to 6.1 L with water. A 2 L volumetric flask is used twice to transfer exactly 4 L of the 10.3 pH solution to a 6 L Erlenmeyer flask. The remaining 10.3 pH solution is diluted to 2 L in a volumetric flask and added to the 6 L Erlenmeyer flask. The 100 ml graduated cylinder is used to add 100±0.1 mL to the 6.0 L Erlenmeyer, and the resulting solution is stirred for 10 minutes.

The resulting product solution appears dark red to brown. Two isotopes of iron are present in the sample preparation in a ratio consistent with that of the standard preparation. The resulting material had a pH of 10.3, which is within the preferred limits of 10.1-10.4. The resultant material had 11.5/11.6 parts per thousand (mg/mL) iron according to SOP 174472, which determines iron through inductively coupled plasma-mass spectroscopy.

The iron sucrose made according to the above-noted process has been found to exhibit an elevated induction of renal heme oxygenase (HO-1). Specifically, intravenous administration of the iron sucrose (FeS) bicarb made according to the above process resulted in elevated renal heme oxygenase (HO-1) after four hours relative to commercially available iron sucrose (FeS) composition sold under the brand name, Venofer®. The results are shown in Table 1 below:

TABLE 1
Kidney mRNA HO-1/GAPDH

| Run# | Control | 4 hr IV FeS, Venofer(R) | 4 hr IV FeS-bicarb |
|---|---|---|---|
| 1 | 0.22 | 1.52 | 3.2 |
| 2 | 0.04 | 1.23 | 2.01 |
| 3 | 0.06 | 1.11 | 1.99 |
| 4 | 0.07 | 2.23 | 2.23 |
| 5 | | 1.86 | 1.86 |
| Avg. | 0.1 | 1.59 | 2.34 |
| Std. Err | 0.04 | 0.21 | 0.23 |

The present inventors surprising discovered through human trials that clinical outcomes can be improved by reducing the amount of stannic protoporfin used. The efficacy exhibited a U-shaped curve and lowering the amount of SnPP was unexpectedly found to increase efficacy. Specifically, the analysis of data in the human clinical trials disclosed herein demonstrate that the amount of stannic protoporfin thought to be effective and desirable of 90 mg was less efficacious compared to a lower dose of 45 mg. In addition, the high purity SnPP used may have also had some role in increasing the effectiveness of the composition. Moreover, the present inventors found that complications related to photosensitivity could be avoided and/or reduced by using a lower dose of SnPP without lowering the effectiveness for preventing post-operative complications related to a surgical procedure. This led the inventors to develop methods for treating patients who were susceptible to photosensitivity with RBT-1.

In one aspect, the present inventors discovered that a lower dose of SnPP could be utilized and that doing so unexpectedly allowed the inclusion of photosensitive patients. The previous literature suggested that photosensitive patients should not be administered SnPP. The present inventors found that the claim methods could use a lower dose of SnPP with photosensitive patients while maintaining the efficacy of RBT-1 for protecting an organ from scheduled insult, e.g., CABG surgery. The present inventors found that the use of sunscreen by a photosensitive patient along with the lower dose of 45 mg SnPP could enable participation in the study.

In another aspect, the present inventors found that patients who were at risk from rehospitalization after CABG surgery could benefit from the lower-dose administration of SnPP. The rate of rehospitalization was decreased by at least 60% relative to placebo, and in cases the rate of rehospitalization was reduced by at least 70%, or more specifically 72%. Other post-operative complications were found to be improved by way of the lower dose administration of SnPP. Those complications include: (a) greater than three days in the intensive care unit, (b) greater than 24 hours on a ventilator, (c) readmission for cardiopulmonary surgery, (d) need for a blood transfusion, (e) new onset post-operative atrial fibrillation (POAF) during hospitalization, or a combination of two or more of (a)-(e). The human patient may be selected from a patient that has an elevated risk post-operative complications including one or more of the above-listed complications after receiving and CABG and/or heart valve surgery.

In certain embodiments, the stannic protoporfin may be administered in a dose of 20-80 mg, more preferably at a dose of 30-70 mg, or a dose of 45 mg. The iron sucrose may be administered at a dose of 190-290 mg, or preferably at a dose of 240 mg. In one embodiment, the stannic protoporfin is administered at a dose of 45 mg and iron compound is iron sucrose is administered at a dose of 240 mg.

In certain embodiments, the iron compound may be present in an aqueous pharmaceutical composition comprising: iron sucrose; bicarbonate; and a pharmaceutically acceptable aqueous carrier. The stannic protoporfin may be present in an aqueous pharmaceutical composition comprising: a total level of impurities of 1.5% or less, as measured by gas chromatography. In one aspect, the preparation of the therapeutically effective composition for administration may comprise combining in an intravenous bag: (a) an aqueous iron pharmaceutical composition comprising: iron sucrose; bicarbonate; and a pharmaceutically acceptable aqueous carrier; and (b) a stannic protoporfin composition having a total level of impurities of 1.5% or less, as measured by gas chromatography.

In certain embodiments, the therapeutically effective amount of stannic protoporfin may be administered at a dose of 0.5-0.8 mg/kg, more preferably 0.6-0.7 mg/kg, and particularly at 0.63 mg/kg. In another aspect, the therapeutically effective amount of iron sucrose may be 2-6 times the amount by weight of stannic protoporfin, or more preferably 3-4 times the amount of stannic protoporfin.

In one aspect, the scheduled or anticipated surgical operation is surgery to an organ. The organ may be heart, kidney, liver or lung. In one aspect, the iron compound and the stannic protoporfin are administered at least 24 hours before the scheduled or anticipated surgical operation to the organ occurs. In one aspect, it is desirable that the iron compound and the stannic protoporfin be administered no more than 48 hrs before the scheduled or anticipated surgical operation to the organ occurs.

In one aspect, the scheduled or anticipated surgical operation may be a surgery comprising coronary artery bypass graft (CABG), cardiac valve, or combined CABG/valve surgery on cardiopulmonary bypass (CPB). In another aspect, the scheduled or anticipated surgical operation is organ transplant surgery. In the case of organ transplantation, the method may reduce or eliminate the need for a blood product to be administered after the scheduled or anticipated surgical operation. This may include whole blood products from a donor.

### COMPARATIVE EXAMPLE

A first composition of Stannic protoporfin was made a comparative example as follows.

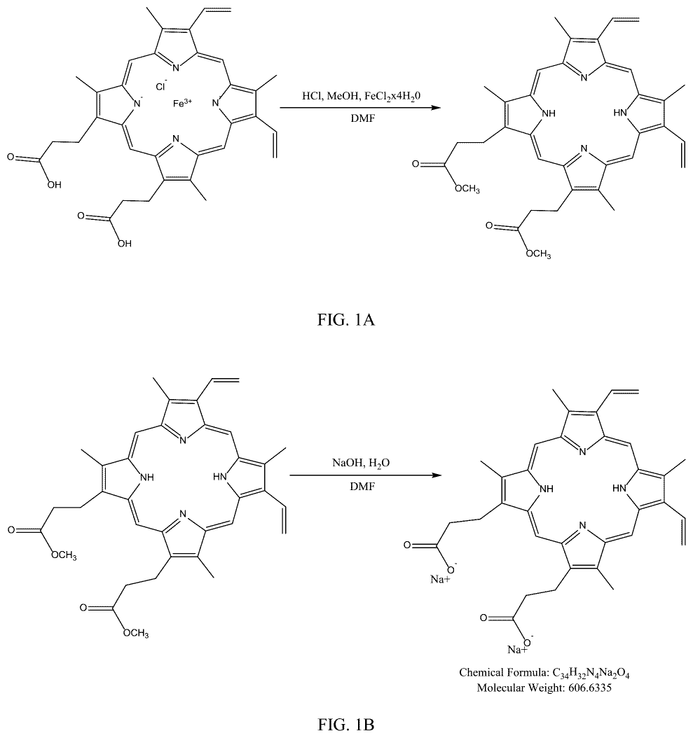

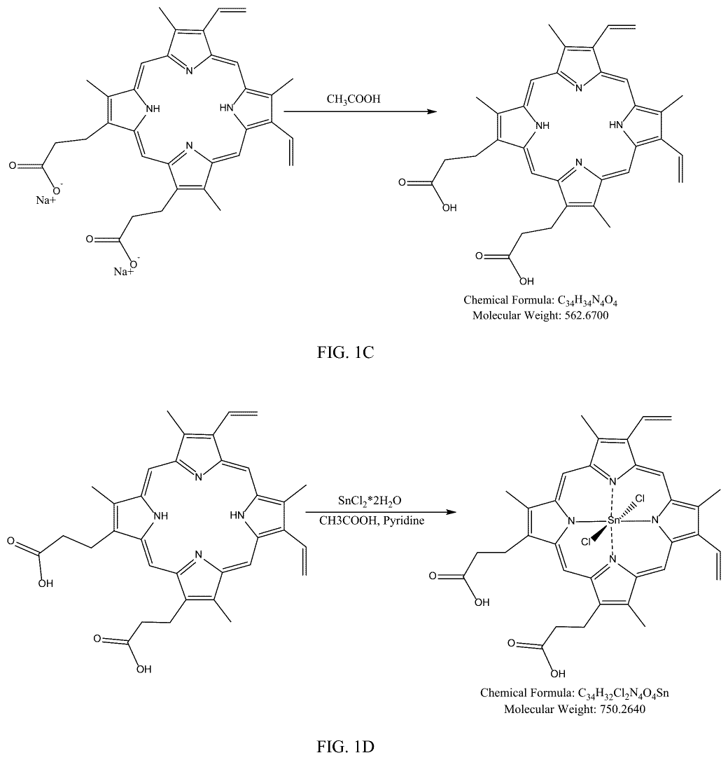

Hemin was dissolved in dimethylformamide at 50-60° C. and filtered. In a separate vessel methanol was saturated with gaseous HCl such that it was heated to 45° C. multiple times. Iron (II) chloride tetrahydrate was then added to the methanol/HCl solution. At this point, the filtered Hemin solution was added by peristaltic pump at ˜25 ml/min such that the temperature was maintained between 40-60° C. Following complete addition of the hemin solution, the reaction was stirred under HCl gas for another 30 minutes. The reaction mixture was then diluted with dichloromethane and washed with water. Aqueous washes are then back-extracted with dichloromethane. The organics were combined and washed once more with water. Following separation, the organics were then split into two portions and purified by silica gel chromatography using a dichloromethane, ethyl acetate gradient. Product fractions were then combined and stripped of solvent by rotary evaporation until ˜25% of the original volume was reached. Ethyl acetate was then added and rotary evaporation was continued and the final slurry was then filtered to isolate a dark purple solid which is protoporphyrin IX dimethyl ester as shown in FIG. 1A.

Protoporphyrin IX dimethyl ester was dissolved in dimethylformamide at 105° C. under inert atmosphere. Aqueous NaOH was added and the temperature was increased to 110° C. The reaction was stirred for 3 hours and allowed to cool. The following day, the reaction was cooled to <6° C. and filtered. The crude protoporphyrin IX disodium salt was washed with cold DMF and acetone. After the product has sufficiently dried on the filter, it was ground with a mortar and pestle and dried in a vacuum over at 80° C. overnight. This step is illustrated in FIG. 1B.

Protoporphyrin IX disodium salt was dissolved in glacial acetic acid under inert atmosphere and stirred at room temperature for at least 24 hours. The reaction was filtered to isolate a red/brown solid. The product was then suspended in 0.5 N acetic acid, stirred overnight and filtered again. Depending on purity of the protoporphyrin IX, one or more recrystallizations was carried out using pyridine at 80° C. followed by cooling to −20° C. and filtration as shown in FIG. 1C.

Glacial acetic acid and pyridine were charged into a 22 L reaction vessel with inert atmosphere at 50° C. Stannous chloride dihydrate was added and stirred to complete dissolution. Protoporphyrin IX was then added and stirred at temperature for a minimum of 48 hours. The resulting solution was then cooled to room temperature, filtered, and rinsed with glacial acetic acid. The final product was then vacuum dried and slurried in hydrochloric acid followed by filtration, deionized water rinse, and finally vacuum drying. This step is shown in FIG. 1D.

### Example 1

A first composition of Stannic protoporfin was made using a two-step process in accordance with an embodiment of the present invention, as follows:

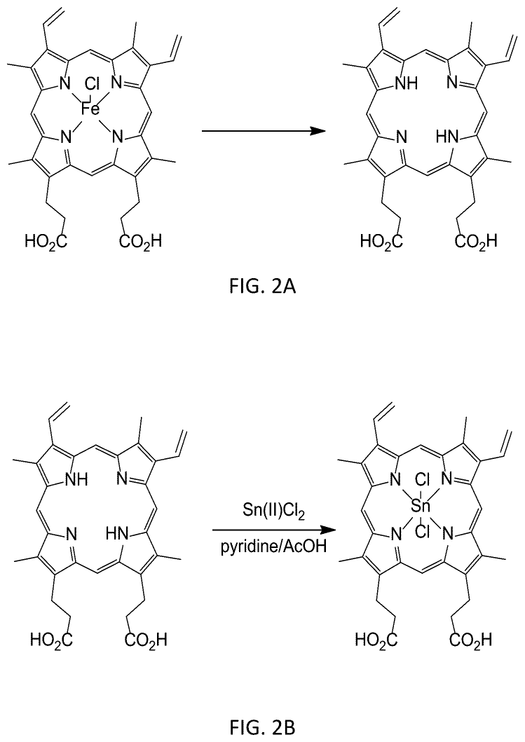

Hemin was dissolved in hot formic acid, then iron powder was added in aliquots over 20 min. The resulting mixture was heated and stirred for 30 min, then filtered through Celite. The filtrate was added to a stirring aqueous solution of NH₄OAc to precipitate the desired product, which was filtered and dried. This crude material was dissolved in hot pyridine and the hot solution was filtered through Celite. The purified product precipitated from the filtrate upon cooing and was recovered by filtration. This corresponds to the first step in this process as shown in FIG. 2A.

Stannous chloride was dissolved in pyridine under inert atmosphere, glacial acetic acid was added and the mixture is heated at 50° C. Protoporphyrin IX was then added and stirred and heated for a minimum of 24 hours and monitored for completion by HPLC. The reaction was cooled to room temperature and filtered. The product was then triturated first with water, then 2 M HCl_{(aq)} and then again with water. An IPC was conducted to determine if a pyridine/AcOH recrystallization, followed by an additional water trituration. The product was then dried to remove residual solvents. This corresponds to the second step in this process as shown in FIG. 2B.

The level of impurities in the Stannic protoporfin was then determined by chromatography. The results are shown in Table 2 below:

TABLE 2
Stannic protoporfin Impurity Profiles

| | | | Total |
|---|---|---|---|
| Batch | Peak | Area % | Impurities % |
| 17-OC-FP-193 | 1 | 0.121 | 1.891 |
| (Comparative | 2 | 0.041 | |
| Example) | 3 | 0.637 | |
| | 4 | 0.678 | |
| | 5 | 0.336 | |
| | SnPP | 98.11 | |
| | 7 | 0.078 | |
| 22-OC-FP-046 | 1 | 0.12 | 0.48 |
| (Example 1) | 2 | 0.06 | |
| | 3 | 0.11 | |
| | SnPP | 99.5 | |
| | PPIX | 0.19 | |

The Stannic protoporfin composition of Example 1 had an almost 4-fold drop in total percent impurities relative to the comparative example. The inventors believe based on preliminary solubility data and other observations, including for example visually observed color, that the composition of Example 1 will likely exhibit unique photodynamic properties, including for example enhanced biological activity, such as enhanced antiviral activity.

### Example 2

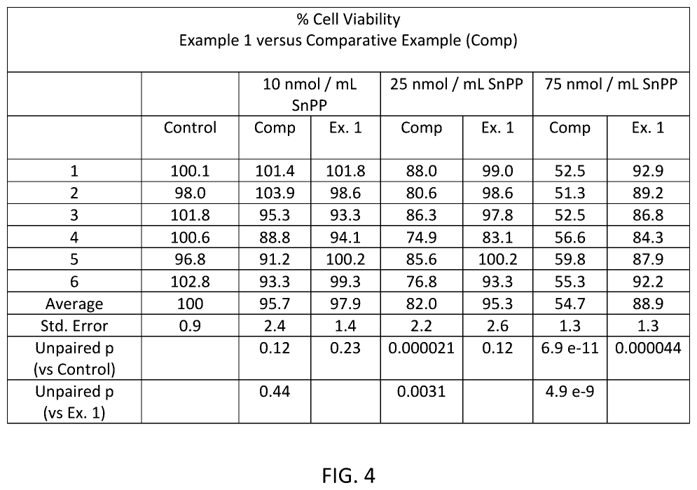

The composition of Example 1 and the Comparative Example were exposed to incubated cultured human tubule cells with different concentrations of SnPP. These data are summarized in Table 3 below. Detailed % viability data is shown in FIG. 4.

TABLE 3
% Viable Cells, n = 6, control 100%

| SnPP (nmol/mL) | Comparative Example | Example 1 |
|---|---|---|
| 75 | 54.7 | 88.9 |
| 25 | 82.0 | 95.3 |
| 10 | 95.7 | 97.9 |

At the highest concentration of SnPP, the amount of cell viability observed for the composition of Example 1 was 88.9% (11% killing) compared to 54.7% cell viability (46% cell killing) for the Comparative Example. These data show that the SnPP composition of Example 1 results in less cell death than the composition made according to the comparative example.

### Example 3

The SnPP composition of Example 1 and the Comparative Example (1 micromole) were combined with iron sucrose (1 mg) to assess biomarkers for oxidative stress. The results are shown in Tables 4A-4B:

TABLE 4A
HO-1/GAPDH mRNA (Cortex)

| | | 4 hr IV FeS + | 4 hr IV FeS + |
|---|---|---|---|
| | Normal | SnPP(Ex1) | SnPP(Comp) |
| 1 | 0.2 | 5.2 | 14.3 |
| 2 | 0.1 | 14.3 | 10.0 |
| 3 | 0.2 | 9.8 | 19.9 |
| Avg. | 0.18 | 9.76 | 14.70 |
| Std. Err | 0.03 | 2.62 | 2.87 |
| unpaired P | | 0.022 | 0.0072 |
| (vs normal) | | | |
| unpaired P | | | 0.27 |
| (vs Ex. 1) | | | |

TABLE 4B
HO-1/GAPDH mRNA (Log 10)

| | | 4 hr IV FeS + | 4 hr IV FeS + |
|---|---|---|---|
| | Normal | SnPP(Ex1) | SnPP(Comp) |
| 1 | −0.78 | 0.7 | 1.2 |
| 2 | −0.9 | 1.2 | 1.0 |
| 3 | −0.6 | 1.0 | 1.3 |
| Avg. | −0.76 | 0.95 | 1.15 |
| Std. Err | 0.07 | 0.13 | 0.09 |
| unpaired P | | 0.00032 | 0.000074 |
| (vs normal) | | | |
| unpaired P | | | 0.27 |
| (vs Ex. 1) | | | |

These data show that the composition of Example 1 exhibits a lower HO-1 induction, which is an indicator of oxidative stress, relative to the composition of the comparative example. These data considered in light of that in Example 2 demonstrate that the SnPP produced according to the method of Example 1, exhibit a unique biomarker profile that suggests a therapeutic use can be achieved with an improved safety profile.

### Example 4

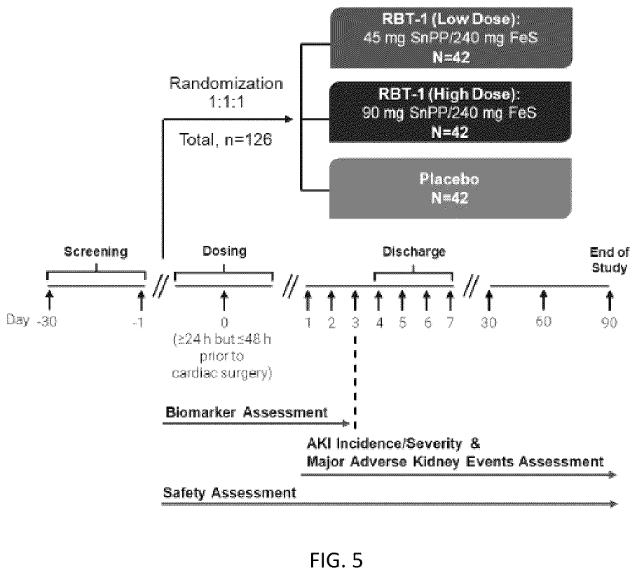

FIG. 5 shows the study design of administering RBT-1 (FeS+SnPP) at a low dose of 45 mg stannic protoporfin (SnPP)/240 mg iron sucrose (FeS) to 42 subjects and at a high dose 90 mg SnPP/240 mg FeS to 42 subjects while observing an addition 42 subjects with a placebo for a total of 126 subjects. The study was a multicenter, double-blind, placebo controlled, Phase 2 study in subjects undergoing coronary artery bypass graft (CABG) and/or cardiac valve surgery on cardiopulmonary bypass. The subjects were administered the doses prior to undergoing the cardiac surgery. The primary objective of the study is to determine the effect of RBT-1 in generating a preconditioning response, measured by a compositive of plasma biomarkers through Day 1 pre-surgery. These biomarkers include heme oxygenase-1 (HO-1), ferritin, and interleukin-10 (IL-10). Key secondary and exploratory objectives of the study include the effect of RBT-1 on the recovery of subjects by measuring days on a ventilator, days in intensive care unit (ICU), hospital length of stay, incidence of acute kidney events (AKI), incidence of major adverse kidney events (MAKE), hospital readmission rate, and safety.

The subjects came from 19 sites across the U.S., Canada, and Australia. The overall study population was not enriched for events. The subjects were randomized at a site level to account for differences in standard of care. The safety population consisted of all subjects who received the study drug. The ITT population consisted of subjects who received the study drug and had biomarker assessments performed per protocol for the primary assessment. The MITT population consisted of ITT subjects who underwent surgery within protocol defined timeframe (all clinical outcomes endpoints are based on this group).

The total number of subjects enrolled in this study was 152 subjects, there were 12 screen fails and 7 withdrawals (5 withdrew pre-infusion). The safety population comprised of 135 subjects, 44 in the placebo group, 45 in the low dose group, and 46 in the high dose group. The ITT population comprised of 132 subjects, 44 in the placebo group, 42 in the low dose group, and 46 in the high dose group. The MITT population comprised of 121 total subjects, 41 in the placebo group, 39 in the low dose group, and 41 in the high dose group. The demographics, baseline characteristics, AKI risk factors, and EuroSCORE, of the subjects in the MITT population can be seen in Tables 5-8.

TABLE 5
MITT Population Demographics

| | | | | Combined |
|---|---|---|---|---|
| | Placebo | Low Dose | High Dose | RBT-1 |
| | (N = 41) | (N = 39) | (N = 41) | (N = 80) |
| Mean Age (yrs) | 65.37 | 64.59 | 66.59 | 65.61 |
| Sex | | | | |
| Female, N (%) | 11 (26.8) | 11 (28.2) | 9 (22.0) | 20 (25.0) |
| Male, N (%) | 30 (73.2) | 28 (71.8) | 32 (78.0) | 60 (75.0) |
| Race | | | | |
| American Indian or | 0 | 0 | 1 (2.4) | 1 (1.3) |
| Alaska Native, N (%) | | | | |
| Black, N (%) | 2 (4.9) | 4 (10.3) | 1 (2.4) | 5 (6.3) |
| Asian, N (%) | 1 (2.4) | 1 (2.6) | 2 (4.9) | 3 (3.8) |
| White, N (%) | 38 (92.7) | 32 (82.1) | 37 (90.2) | 69 (86.3) |
| Other, N (%) | 0 | 2 (5.1) | 0 | 2 (2.5) |
| Weight (kg), | 88.7 | 97.4 | 90.9 | 94.0 |
| Mean (min, max) | (64, 132) | (51, 142) | (57, 150) | (51, 150) |
| BMI (kg/m²), | 29.7 | 32.8 | 30.2 | 31.4 |
| Mean (min, max) | (19, 45) | (18, 48) | (20, 49) | (18, 49) |

TABLE 6
Baseline Characteristics

| | | | | Combined |
|---|---|---|---|---|
| | Placebo | Low Dose | High Dose | RBT-1 |
| | (N = 41) | (N = 39) | (N = 41) | (N = 80) |
| Time of Infusion Before Surgery | | | | |
| N | 41 | 39 | 41 | 80 |
| Mean (hrs) | 38.6 | 38.6 | 38.4 | 38.5 |
| Surgery Type | | | | |
| CABG Alone, N (%) | 20 (48.8) | 20 (51.3) | 24 (58.4) | 44 (55.0) |
| Valve Alone, N (%) | 8 (19.5) | 13 (33.3) | 9 (22.0) | 22 (27.5) |
| CABG + Valve, N (%) | 13 (31.7) | 6 (15.4) | 8 (19.5) | 14 (17.5) |
| Duration of Surgery | | | | |
| N | 41 | 39 | 41 | 80 |
| Mean (hrs) | 4.941 | 5.051 | 4.929 | 4.988 |
| Time on Pump | | | | |
| N | 41 | 39 | 41 | 80 |
| Mean (hrs) | 1.946 | 1.952 | 1.996 | 1.974 |

TABLE 7
AKI Risk Factors

| | | | | Combined |
|---|---|---|---|---|
| | Placebo | Low Dose | High Dose | RBT-1 |
| Risk Factor - N (%) | (N = 41) | (N = 39) | (N = 41) | (N = 80) |
| 1. Combined CABG and valve surgery | 13 (31.7) | 6 (15.4) | 8 (19.5) | 14 (17.5) |
| 2. Previous cardiac surgery with | 0 | 1 (2.6) | 1 (2.4) | 2 (2.5) |
| sternotomy | | | | |
| 3. Documented heart failure (NHYA III/IV) | 2 (4.9) | 3 (7.7) | 5 (12.2) | 8 (10) |
| within 1 year prior to surgery | | | | |
| 4. LVEF ≤ 35% | 2 (4.9) | 3 (7.7) | 4 (9.8) | 7 (8.8) |
| 5. Congestive heart failure | 6 (14.6) | 5 (12.8) | 7 (17.1) | 12 (15) |
| 6. Diabetes mellitus requiring insulin | 3 (7.3) | 6 (15.4) | 8 (19.5) | 14 (17.5) |
| 7. T2D with albuminuria (urine albumin > | 0 | 0 | 0 | 0 |
| 300 mg/g of creatinine, as documented | | | | |
| in medical history) | | | | |
| 8. Pre-operative anemia (hemoglobin < 10 | 1 (2.4) | 1 (2.6) | 0 | 1 (1.3) |
| g/dl upon screening) | | | | |
| 9. Currently hospitalized for management | 8 (19.5) | 5 (12.8) | 12 (29.3) | 17 (21.3) |
| of cardiac or pulmonary disease | | | | |
| 10. eGFR ≥ 20 to < 30 mL/min/1.73 m² | 2 (4.9) | 0 | 0 | 0 |
| 11. eGFR ≥ 30 to < 60 mL/min/1.73 m² | 4 (9.8) | 13 (33.3) | 13 (31.7) | 26 (32.5) |
| 12. Age ≥ 65 years | 23 (56.1) | 23 (59.0) | 25 (61.0) | 48 (60.0) |

TABLE 8
EuroSCORE

| | Placebo | Low Dose | High Dose | Combined RBT-1 |
|---|---|---|---|---|
| EuroSCORE | (N = 41) | (N = 39) | (N = 41) | (N = 80) |
| Mean (Min, Max) | 1.89 (0.56, 9.73) | 2.78 (0.50, 17.35) | 2.39 (0.50, 9.13) | 2.58 (0.50, 17.35) |
| Median | 1.47 | 1.05 | 1.93 | 1.52 |
| Low Risk (<2.99), N (%) | 37 (90) | 30 (77) | 30 (73) | 60 (75) |
| Medium Risk (3 to 5.99), N | 2 (5) | 4 (10) | 9 (22) | 13 (16) |
| (%) | | | | |
| High Risk (>5.99), N (%) | 2 (5) | 5 (13) | 2 (5) | 7 (9) |

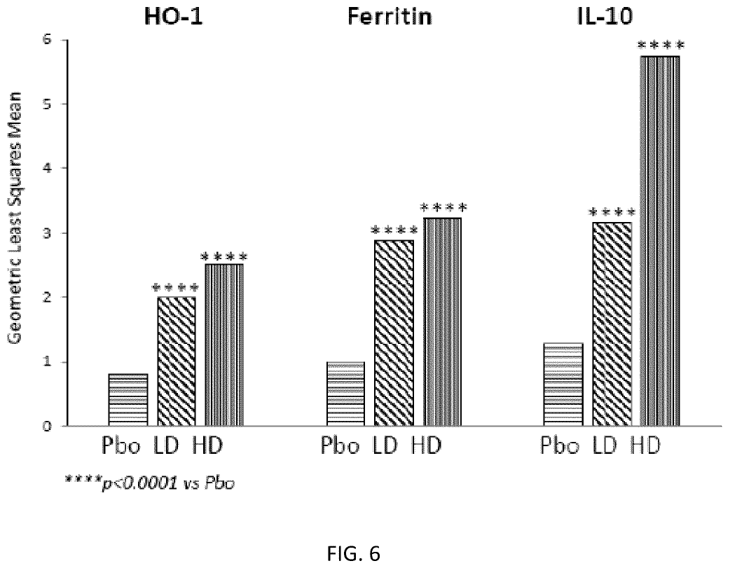

FIG. 6 shows a graph demonstrating the statistically significant increase in anti-inflammatory and antioxidant biomarkers of cytoprotective preconditioning with RBT-1 for the MITT population (p<0.0001). The biomarkers assessed were interleukin-10 (IL-10), heme oxygenase-1 (HO-1), and ferritin. The composite biomarker response for the data shown in FIG. 6 is shown in Table 5.

TABLE 9
Composite Biomarker Response

| | Placebo | Low Dose | High Dose |
|---|---|---|---|
| | (N = 44) | (N = 42) | (N = 46) |
| Mean | 0.98 | 2.65 | 3.62 |
| P-value vs Pbo | | <0.0001 | <0.0001 |
| P-value LD vs HD | | | 0.0046 |

All of the subsequent analyses have been conducted on the MITT population as predefined.

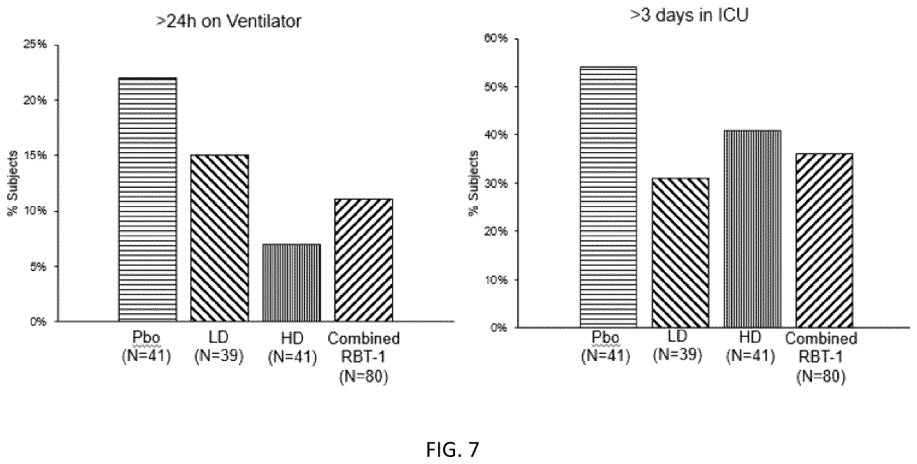

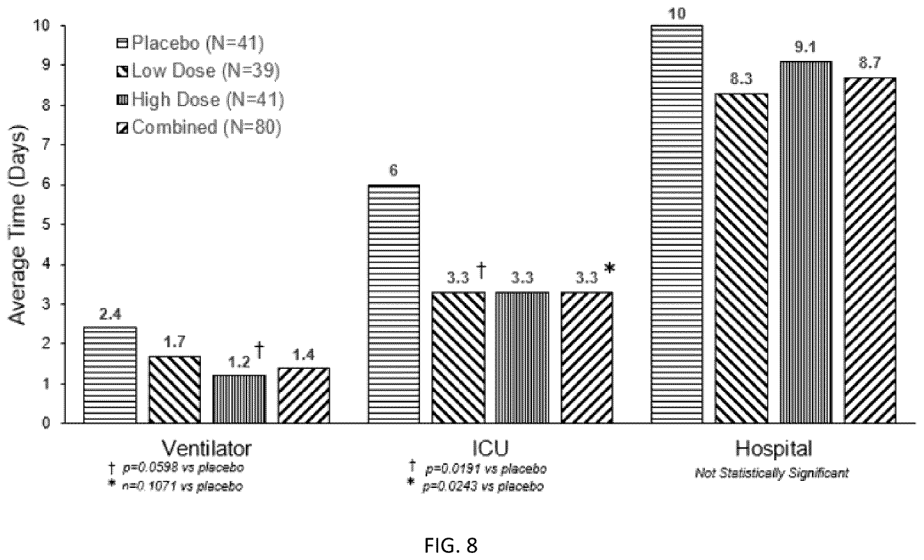

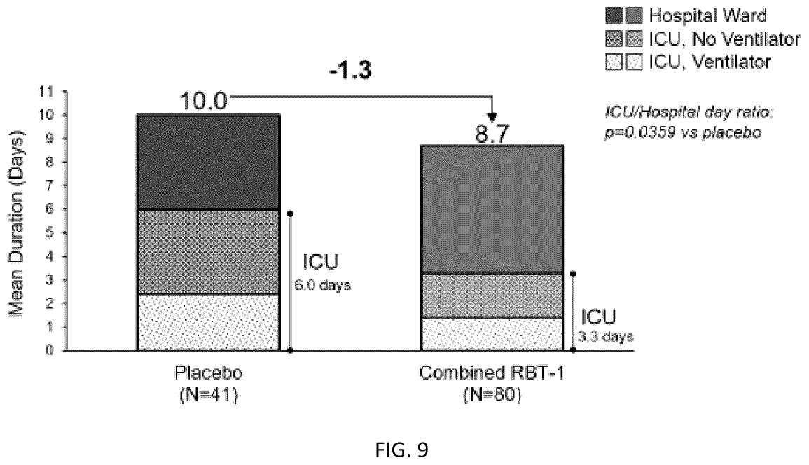

FIG. 7 shows a graph of the extended time on the ventilator and in the ICU spent by subjects in each group. The graph illustrates a decline in the percentage of subjects that required a ventilator post-surgery between the placebo group and the low and high dose groups with the high dose group having the lowest percentage. FIG. 7 additionally shows a decline in the percentage of subjects that required greater than 3 days in the ICU post-surgery with the low dose group having the smallest percentage. FIG. 8 shows a graph of the average time of subjects in each group spent on a ventilator, in the ICU, and in the hospital. The graph shows a reduction in ventilator, ICU, and hospital time in patients treated with RBT-1. An overall improvement in mean composition of hospitalization in patients treated with RBT-1 can be seen in FIG. 9.

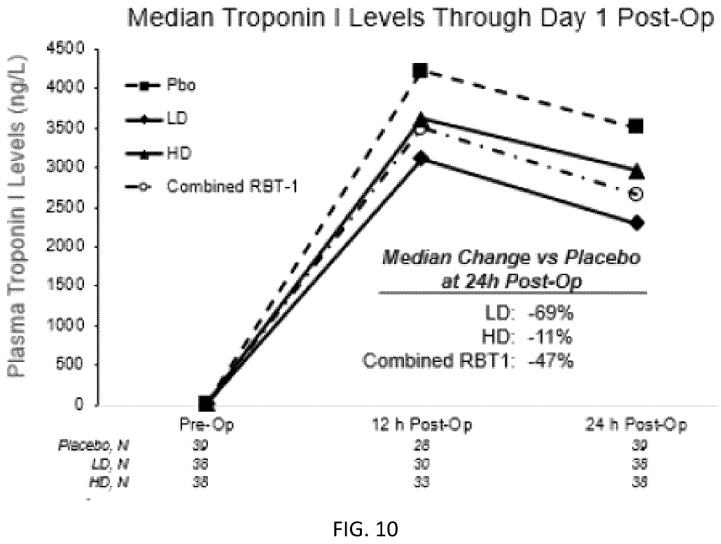

The low dose of RBT-1 group exhibited the lowest median levels of Troponin I levels while the high dose group additionally exhibited lower levels than that of the placebo group. There was a clinically meaningful reduction in Troponin I levels (47%) for the combined RBT-1 groups as compared to the placebo group. FIG. 10 shows the increase in plasma troponin I from pre-operative baseline to 12 hours (left series) and 1 day (right series) after cardiac surgery. The analysis population, derived from the modified intent-to treat population, excluded patients who had undergone mitral valve repair or replacement, ablation, or septal myectomy due to the expected significant increase in troponin I levels following these major surgeries.

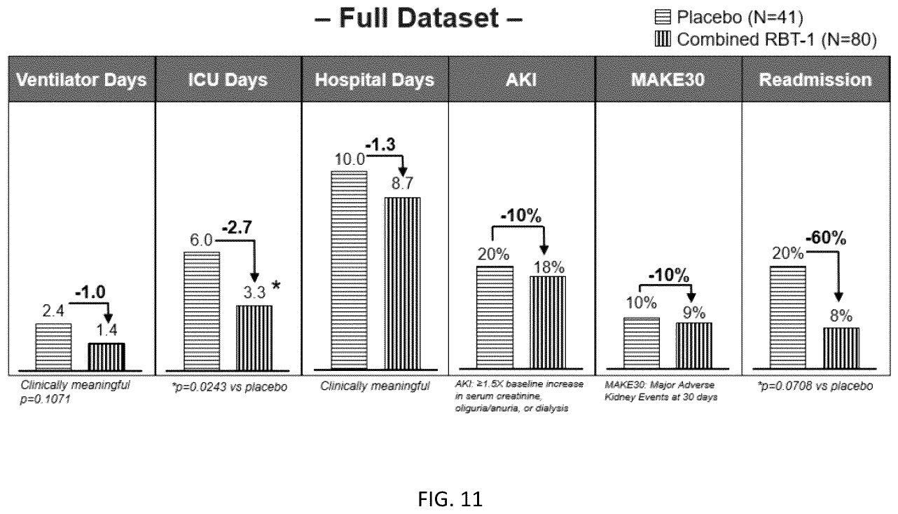

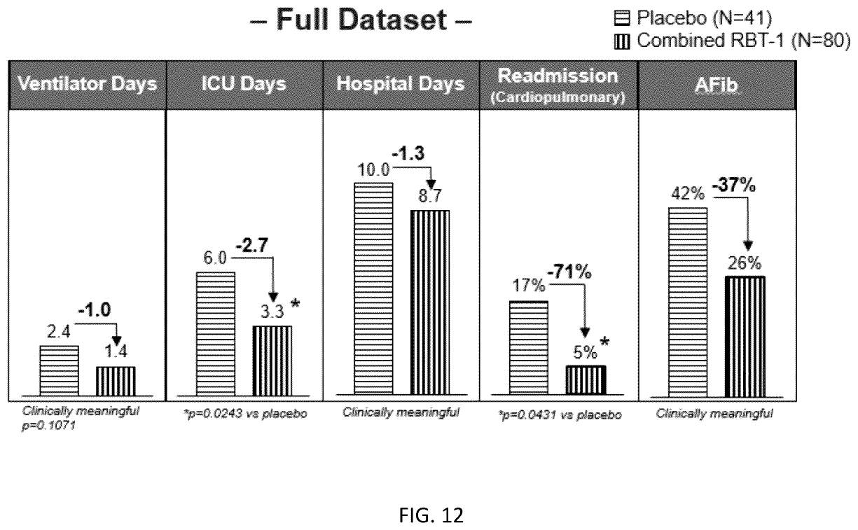

In FIG. 11 and FIG. 12, graphs illustrate the improvement of postoperative outcomes of the combined RBT-1 groups over the placebo group in measurements such as ventilator days, ICU days, hospital days, AKI, major adverse kidney events at 30 days (MAKE30), readmission, readmission (cardiopulmonary), and Atrial Fibrillation (AFib). There is a clinically meaningful improvement in subjects treated with RBT-1 over the placebo in ventilator days (−1 day), length of hospital stay (−1.3 days), AKI rate (−10%), and in AFib rates (−37%).

There was a statistically significant (−2.7 days, p<0.03) reduction in days in the ICU. A statistically significant (−71%, p<0.05) reduction in 30-day hospital readmission rates due to cardiopulmonary causes and a 60% reduction in all-cause readmissions. The data also indicated a generally well-tolerated safety profile.

A win ratio for the combination of high and low doses of RBT-1 was calculated on the data collected in the trial. A win ratio is a method for examining composite endpoints and has since been widely adopted in cardiovascular (CV) trials. Improving upon conventional methods for analyzing composite endpoints, the win ratio accounts for relative priorities of the components and allows the components to be different types of outcomes. The win ratio is further described by Redfors B. et al. The win ratio approach for composite endpoints: practical guidance based on previous experience. Eur Heart J. 2020 Dec. 7; 41 (46): 4391-4399. doi: 10.1093/eurheartj/ehaa665. PMID: 32901285.

Table 10-13 illustrate the win ratio calculation for the trial. In an analysis of composite endpoint of death, ICU days, ventilator days, atrial fibrillation rates, hospital days and hospital admission rates using the win ratio method, a highly statistically significant benefit was observed among the treated groups (win ratio 1.63, p<0.02). These positive topline data provide strong support for RBT-1's potential to reduce the risk of multiorgan injury and thereby improve post-operative outcomes in patients undergoing cardiothoracic surgery. These data suggest that RBT-1 can provide wide protection against organ damage, and has the potential to reduce post-operative complications, lengths of stay, and costs of care.

TABLE 10

| | HD + LD | Placebo | p-value (2-sided) |
|---|---|---|---|
| Death (%) | 3.75 | 7.317073171 | 0.392277603 |
| ICU Days (mean) | 3.2875 | 6 | 0.022492274 |
| Vent Days (mean) | 1.4375 | 2.43902439 | 0.104440839 |
| Afib (%) | 26.25 | 41.46341463 | 0.087901013 |
| Readmission (%) | 10 | 24.3902439 | 0.035253013 |
| Hosp Days (mean) | 8.7 | 9.975609756 | 0.800942124 |

TABLE 11

| | Died | ICU | Vent | AFib | Readmission | Hosp Days |
|---|---|---|---|---|---|---|
| Win | 231 | 1554 | 26 | 104 | 43 | 55 |
| Loss | 114 | 932 | 13 | 69 | 17 | 87 |

TABLE 12

| | Win | Tie | Loss |
|---|---|---|---|
| Pairs | 2013 | 35 | 1232 |

TABLE 13

| | Win Ratio | 1-sided p-value |
|---|---|---|
| Result | 1.63392857 | 0.0157 |

Several treatment comparisons stratified by the weight category of subjects were done to determine benefits of varying per-kg dosing (mg/kg) and to explore a minimum viable dose. Unexpectedly, the drug benefit increased at lower doses. In general, the ideal range is 20-70 mg based on a 70 kg patient, preferably 28-63 mg, or 35-63 mg.

To define the categories, tertials were used (i.e., about ⅓ of the patients in each category). “Low weight” is less than about 81 kg (mean of 72 kg), “medium weight” is between about 81 kg and 99 kg (mean of 90 kg) and “high weight” is greater than about 99 kg (mean of 115 kg). Table 14 shown below shows the win-ratio results stratified by the weight category. Using the mean weights within categories, the results displayed in order of per-kg dosing show more of a U-shaped relationship. Once a certain threshold of dosage is crossed, the effectiveness of the dose declines and the benefits of the dose declines.

TABLE 14
Win-Ratio

| | Weight | Dose | Win- | |
|---|---|---|---|---|
| Comparison | Category | (mg/kg) | ratio | 95% CI |
| LD vs Placebo | High | 0.4 | 2.0 | 0.8, 5.1 |
| LD vs Placebo | Medium | 0.5 | 1.6 | 0.6, 4.30 |
| LD vs Placebo | Low | 0.6 | 2.5 | 0.8, 7.4 |
| HD vs Placebo | High | 0.8 | 1.8 | 0.7, 5.0 |
| HD vs Placebo | Medium | 1.0 | 1.4 | 0.5, 3.8 |
| HD vs Placebo | Low | 1.3 | 1.0 | 0.3, 3.0 |

The 95% confidence interval (CI) results are rough estimates to illustrate that these subgroup analyses have a small sample size, and the Win-Ratios aren't very precise. However, these results overall suggest a peak effect around 0.6 or 0.7 mg/kg. For example, a 100 kg person that corresponds to a suggested dosing of 63 mg, although the data also suggests that 63 mg might be a bit too high for a lighter patient. In one embodiment, the dose administered to the patient is a function of the patient's weight to improve the benefits of the dose.

Adverse events of interest in the trial were recorded. An overview of treatment-emergent adverse events (TAEs) are shown in Table 15 and TAEs of interest for the safety population are shown in Table 16.

TABLE 15
An overview of Treatment-Emergent Adverse Events (TEAEs)

| | Placebo | LD | HD | Combined RBT-1 |
|---|---|---|---|---|
| | (N = 44) | (N = 45) | (N = 46) | (N = 91) |
| Subjects with any | 40 (90.9) | 39 (86.7) | 44 (95.7) | 83 (91.2) |
| TEAE | | | | |
| Maximum | 7 (15.9) | 11 (24.4) | 15 (32.6) | 26 (28.6) |
| Severity of Mild | | | | |
| Maximum | 18 (40.9) | 17 (37.8) | 17 (37.0) | 34 (37.4) |
| Severity of | | | | |
| Moderate | | | | |
| Maximum | 15 (34.1) | 11 (24.4) | 12 (26.1) | 23 (25.3) |
| Severity of Severe | | | | |
| Subjects with at | 6 (13.6) | 12 (26.7) | 18 (39.1) | 30 (33.0) |
| least one | | | | |
| Treatment- | | | | |
| Related TEAE | | | | |
| Excluding | 5 (11.4) | 7 (15.6) | 8 (17.4) | 15 (16.5) |
| Adjudicated | | | | |
| Photosensitivity | | | | |
| Subjects with at | 18 (40.9) | 13 (28.9) | 22 (47.8) | 35 (38.5) |
| least one Serious | | | | |
| TEAE | | | | |
| Subjects | 0 | 0 | 0 | 0 |
| Discontinued due | | | | |
| to TEAE | | | | |
| Died on Study | 3 (6.8) | 1 (2.2) | 2 (4.3) | 3 (3.3) |
| Cause of Deaths | Sepsis | Acute | Cardiogenic | |
| | Stroke | respiratory | shock | |
| | Cardiac arrest | failure | CO₂ retention | |
| | | | from chronic | |
| | | | lung disease | |

TABLE 16
TEAEs of Interest - Safety Population

| | Placebo | LD | HD | Combined RBT-1 |
|---|---|---|---|---|
| | (N = 44) | (N = 45) | (N = 46) | (N = 91) |
| Atrial Fibrillation, N (%) | 17 (38.6) | 11 (24.4) | 10 (21.7) | 21 (23.1) |
| Anemia, N (%) | 11 (25.0) | 8 (17.8) | 6 (13.0) | 14 (15.4) |
| Hypervolemia, N (%) | 10 (22.7) | 4 (8.9) | 5 (10.9) | 9 (9.9) |
| Leukocytosis, N (%) | 6 (13.6) | 3 (6.7) | 4 (8.7) | 7 (7.7) |

Occurrences of photosensitivity were recorded and are shown in Table 13. Three surgeries were postponed due to photosensitivity of the subjects. Each postponed surgery due to photosensitivity occurred for high dose subjects and were additionally exposed to the sun for a prolonged period of time post-infusion.

TABLE 17
Photosensitivity AEs Considered Related by Site

| | Placebo | LD | HD |
|---|---|---|---|
| | (N = 44) | (N = 45) | (N = 46) |
| Photosensitivity, N (%) | 1 (2) | 5 (11) | 10 (22) |
| Day of Onset Post-Infusion, Median | 14.0 | 2.0 | 2.0 |
| Day of Onset Post-Infusion, Median | — | 4.0 | 8.0 |

### Example 5

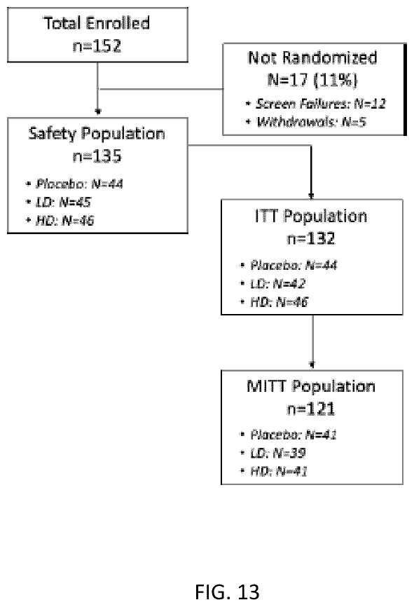

152 patients were enrolled at 19 sites across the US, Canada, and Australia and 135 patients were randomized. The safety population consisted of patients, all of whom received study drug. Of those, 132 (98%) patients had biomarker measurements collected (ITT population). From the ITT population, 121 patients had surgery on time (24 to 48 hours after infusion) and constituted the MITT population, for whom secondary endpoints and clinical outcomes were evaluated. FIG. 13 shows enrollment and patient populations. The total number of patients enrolled and randomized are shown. Distribution of patients by dose group are provided for each patient population. Reasons for screen failures included high serum ferritin (N=5), receipt of IV iron prior to planned infusion (N=1), acute organ injury/unstable organ function (n=4), hypersensitivity to tin-based products (n=1), and change in surgery location (n=1). Five patients withdrew from the study prior to treatment; two patients withdrew from the study post-treatment (one due to postponed surgery and one due to difficulty with blood draws).

In the MITT population, 41 patients received placebo (normal saline), 39 patients received low-dose RBT-1 (45 mg SnPP/240 mg FeS), and 41 patients received high-dose RBT-1 (90 mg SnPP/240 mg FeS). Baseline characteristics of the randomized patients were similar among groups (Table 18). Time between infusion and start of surgery, as well as time on CPB, were also similar between the three groups.

TABLE 18

| | Placebo | Low-dose | High-dose |
|---|---|---|---|
| | (N = 41) | (N = 39) | (N = 41) |
| Age (years), Mean (min, max) | 65 (19, 81) | 65 (46, 82) | 67(37, 86) |
| Sex Male, N (%) | 30(73) | 28(72) | 32(78) |
| Race, N (%) | | | |
| American Indian | 0 | 0 | 1(2) |
| Black | 2(5) | 4(10) | 1(2) |
| Asian | 1(2) | 1(3) | 2(5) |
| White | 38(93) | 32(82) | 37(90) |
| Other | 0 | 2(5) | 0 |
| Weight (kg), Mean (min, max) | 89(64, 132) | 98(51, 142) | 91(57, 150) |
| BMI (kg/m2), Mean (min, max) | 30(19, 45) | 33(18, 48) | 30(20, 49) |
| EuroSCORE II, Mean (min, max) | 2.1(1, 10) | 2.8(1, 17) | 2.4(1,9) |
| Low Risk (<3), N (%) | 35(85) | 31(80) | 31(76) |
| Medium Risk (3 to 6), N (%) | 4(10) | 3(8) | 8(20) |
| High Risk (≥6), N (%) | 2(5) | 5(13) | 2(5) |
| ≥3 AKI Risk Factors, * N (%) | 39 ± 9.9 | 39 ± 9.2 | 38 ± 9.4 |
| Time infusion to surgery, Mean ± SD (hrs) | | | |
| Surgery Type | | | |
| CABG alone, N (%) | 20(49) | 20(51) | 24(59) |
| Valve alone, N (%) | 7(17) | 13(33) | 9(22) |
| CABG + Valve, N (%) | 14(34) | 6(15) | 8(20) |
| Time on CPB, Mean ± SD (hrs) | 2.0 ± 1.0 | 2.0 ± 0.8 | 2.0 ± 1.2 |

### Primary Outcome

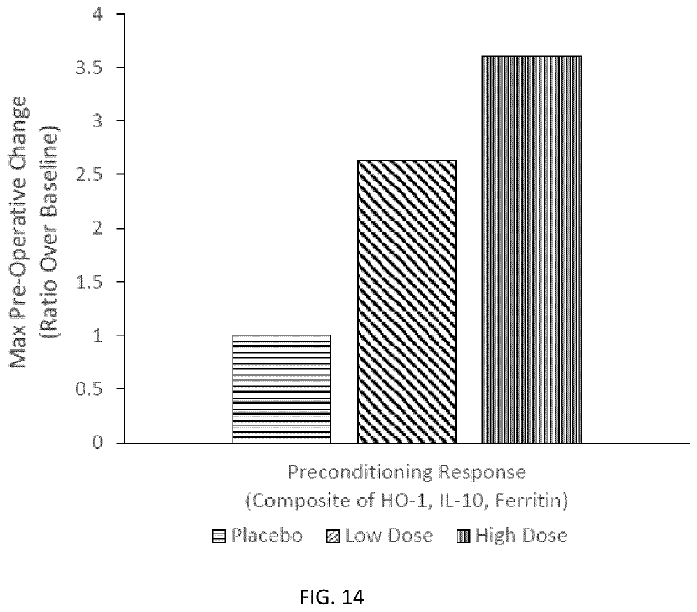

The mean composite of the maximum pre-operative change from baseline in HO-1, IL-10, and ferritin was 1.00 (95% CI: 0.86, 1.17) in the placebo group, 2.63 (95% CI: 2.25, 3.07) in the low dose RBT-1 group, and 3.60 (95% CI: 3.10, 4.18) in the high-dose RBT-1 group, FIG. 14, p<0.0001 for both comparisons. FIG. 14 shows the primary endpoint-preconditioning response. The preconditioning response is shown as the geometric least squares mean for the ratio of the maximum pre-operative change over baseline in a composite of heme oxygenase-1 (HO-1), interleukin-10 (IL-10), and ferritin; *** p<0.001 vs placebo.

### Secondary Endpoints

AKI incidence and sustained reduction in urine output were numerically lower with both doses of RBT-1 but were not significantly different compared with placebo (Table 19). There were no significant changes in the composite of renal tubular injury biomarkers (Table 19), and these biomarkers did not correlate with the maximum change in serum creatinine.

TABLE 19

| | Placebo | Low-dose | P value (vs. | High-dose | P value (vs. |
|---|---|---|---|---|---|
| | (N = 41) | (N = 39) | placebo) | (N = 41) | placebo) |
| AKI (%) ** | 8(19.5) | 7(17.9) | >0.999 | 7(17.1) | >0.999 |
| Sustained reduction | 4(9.8) | 2(5.1) | 0.676 | 2(4.9) | 0.676 |
| urine output, N(%) *** | | | | | |
| Tubular injury | 6.10 (3.96, 9.39) | 10.84 (6.92, 16.98) | 0.068 | 7.89 (5.09, 12.23) | 0.395 |
| biomarker response, | | | | | |
| GLSM* (95% CI) | | | | | |

*GLSM, geometric least squares mean of the ratio of max Post-Op value over Baseline.
** AKI is defined as a ≥1.5 X serum creatinine increase from baseline, sustained reduction in urinary output, or initiation of dialysis post-cardiac surgery through Day 5.
*** Sustained reduction in urinary output was defined as a reported adverse event (AE) of oliguria, anuria, or “sustained” reduction in urine output post-cardiac surgery through Day 5.

### Other Prespecified Endpoints

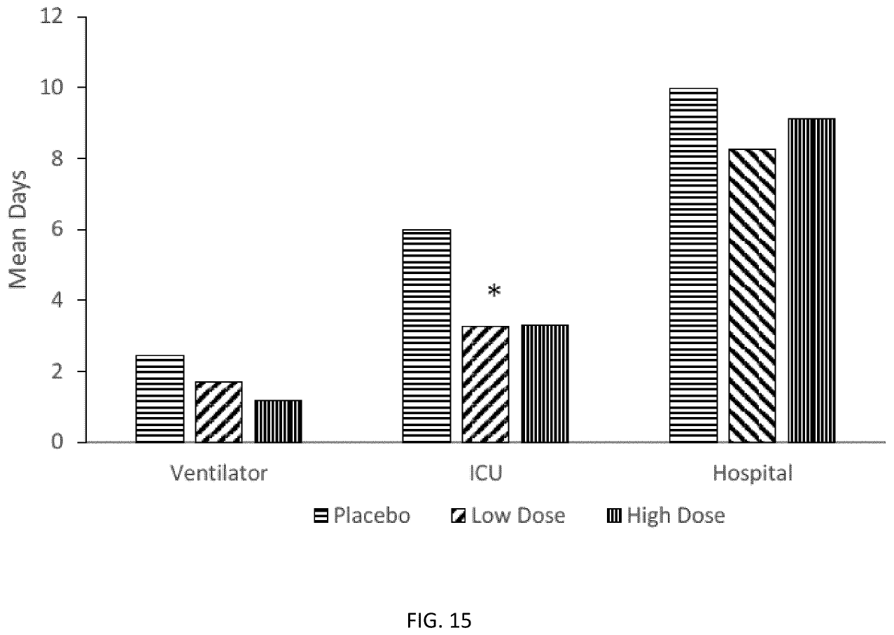

Hospitalization course was assessed by time on ventilator and length of stay (LOS) in the ICU and hospital. FIG. 15 shows Ventilator, ICU, and Hospital Course. The mean time (days) on ventilator, in the ICU, and in the hospital is shown for each treatment group; *p=0.02 vs placebo. The mean time on ventilator was 2.4 days, 1.7 days, and 1.2 days in the placebo, low-dose RBT-1, and high-dose RBT-1 groups (p=0.428 and p=0.060 vs placebo, respectively). The mean LOS in ICU was 6 days in the placebo group and 3.3 days in the low dose and high-dose RBT-1 groups (p=0.019 and p=0.128 vs placebo, respectively). The mean LOS in hospital was 10.0 days, 8.3 days, and 9.1 days in the placebo, low-dose RBT-1, and high dose RBT-1 groups, respectively (p=0.744 and p=0.918 vs placebo, respectively).

Post-operative complications included three deaths (7.3%) in the placebo group, one death (2.6%) in the low-dose RBT-1 group and two deaths (4.9%) in the high-dose RBT-1 group (Table 20). Dialysis for AKI was needed in one patient (2.4%) in the placebo group but none in the RBT-1 groups. Post-operative atrial fibrillation developed in 17 (42%), 12 (31%), and 10 (24%) patients in the placebo, low-dose RBT-1, and high-dose RBT-1 groups, respectively (Table 20). Hypervolemia was diagnosed in 10 (24%), 3 (8%), and 4 (10%) of patients in the placebo, low-dose RBT-1, and high-dose RBT-1 groups (Table 20). Of note, the change from baseline in post-operative troponin I levels 1-day post-surgery was reduced by 63% in the low dose RBT-1 group and 30% in the high-dose RBT-1 group (p=0.016 for low-dose RBT-1 vs placebo). Patients were evaluated for MAKE at 30, 60, and 90 days. The number of patients with MAKE was low as expected with this unenriched population, and no statistical difference between groups were observed (Table 20).

Finally, seven patients (17%) were readmitted to the hospital at 30-days post-discharge for a cardiopulmonary diagnosis in the placebo group compared with two patients (5%) each in the low-dose and high dose RBT-1 groups (Table 20). At 60- and 90-days post-discharge, eight patients (21%) in the placebo group required cardiopulmonary readmissions in contrast to two patients (5%) each in the low-dose and high-dose RBT-1 groups. All-cause readmissions showed similar results (Table 20).

TABLE 20

| | | | Low- | | RR | P value | High- | | RR | P value |
|---|---|---|---|---|---|---|---|---|---|---|
| | Placebo | | dose | | 95% | (vs. | dose | | 95% | (vs. |
| | (N = 41) | | (N = 39) | | CI | placebo) | (N = 41) | | CI | placebo) |
| Death, N (%) | 3 | (7.3) | 1 | (2.6) | | | 2 | (4.9) | | |
| AKI with dialysis, N (%) | 1 | (2.4) | 0 | | | | 0 | | | |
| MAKE 30, N (%) | 4 | (9.8) | 1 | (2.6) | | | 4 | (9.8) | | |
| MAKE 60, N (%)¶ | 2 | (5.0) | 1 | (2.6) | | | 3 | (7.3) | | |
| Atrial fibrillation, N (%) | 17 | (41.5) | 12 | (30.8) | 0.74 | 0.359 | 10 | (24.4) | 0.59 | 0.158 |
| | | | | | (0.36, | | | | (0.28, | |
| | | | | | 1.36) | | | | 1.13) | |
| Hypervolemia, N (%) | 10 | (24.4) | 3 | (7.7) | 0.32 | 0.067 | 4 | (9.8) | 0.4 | 0.141 |
| | | | | | (0.07, | | | | (0.07, | |
| | | | | | 1.01) | | | | 1.15) | |
| 30 days | 7 | (18.4) | 2 | (5.3) | 0.29 | 0.153 | 2 | (5.1) | 0.28 | 0.087 |
| Cardiopulmonary | | | | | (0.03, | | | | (0.03, | |
| readmissions, N (%)*‡ | | | | | 1.00) | | | | 1.14) | |
| 30 days All | 8 | (21.1) | 2 | (5.3) | 0.25 | 0.086 | 4 | (10.3) | 0.49 | 0.087 |
| readmissions, N (%)*‡ | | | | | (0.03, | | | | (0.08, | |
| | | | | | 1.00) | | | | 1.53) | |
| 60 Cardiopulmonary | 8 | (21.1) | 2 | (5.3) | 0.25 | 0.086 | 2 | (5.1) | 0.24 | 0.047 |
| readmissions, N (%)*‡ | | | | | (0.03, | | | | (0.03, | |
| | | | | | 1.00) | | | | 0.98) | |
| 60 days all | 10 | (26.3) | 3 | (7.9) | 0.30 | 0.065 | 5 | (12.8) | 0.49 | 0.160 |
| readmissions, N (%)*‡ | | | | | (0.07, | | | | (0.13, | |
| | | | | | 0.96) | | | | 1.29) | |

¶MAKE 60 and 90 results were identical.
*Subjects who died during index hospitalization are excluded from readmission analysis; total sample size for placebo, LD, and HD is 38, 38, and 39 patients, respectively.
‡Cardiopulmonary readmissions and All readmissions results were identical at 60 days and 90 295 days.

### Post Hoc Analysis

Given the suggested multi-organ benefit of RBT-1, we explored the effects of RBT-1 in a post hoc composite analysis (win ratio) wherein clinical outcomes were assessed in rank order of severity (death, AKI requiring dialysis, ICU days, 30-day cardiopulmonary readmission, atrial fibrillation, and hospital days). The win ratio was 2.02 (p=0.004) in the low-dose RBT-1 group and 1.34 (p=0.131) in the high-dose RBT-1 group (Table 21 A-B), showing that patients treated with RBT-1 had improved outcomes compared to those treated with placebo.

TABLE 21 A

| | Wins | Tie | Loss | Win Ratio (95% CI) | p-value |
|---|---|---|---|---|---|
| Low dose RBT-1 | 1056 | 21 | 522 | 2.02 (1.19, 3.44) | 0.004 |
| High dose RBT-1 | 952 | 16 | 713 | 1.34 (0.80, 2.23) | 0.131 |

Table 21 B

| | | AKI | | 30-Day | | |
|---|---|---|---|---|---|---|
| | | Requiring | ICU | Cardiopulmonary | Atrial | Hospital |
| | Death | Dialysis | Days | Readmission | Fibrillation | LOS |
| Low-dose RBT-1 | | | | | | |
| Win | 114 | 1 | 817 | 50 | 38 | 46 |
| Loss | 38 | 0 | 388 | 8 | 42 | 46 |
| High-dose RBT-1 | | | | | | |
| Win | 117 | 2 | 734 | 34 | 40 | 25 |
| Loss | 76 | 0 | 544 | 18 | 21 | 54 |

¶ MAKE 60 and 90 results were identical.

*Subjects who dies during index hospitalization are excluded from readmission analysis; total sample size for placebo, LD, and HD is 38, 38, and 39 patients, respectively.

‡ Cardiopulmonary readmissions and All readmissions results were identical at 60 days and 90 295 days.

Win ratio, based on the Finkelstein-Schoenfeld method, derived from rank order analysis of death, AKI requiring dialysis, ICU days, 30-day cardiopulmonary readmission, atrial fibrillation, and hospital length of stay and comparisons made between each patient on placebo and each patient on low-dose RBT-1 and high-dose RBT-1. The cells in the upper table indicate the overall number of wins and losses across all endpoints, and the cells in the lower table indicate the number of wins and losses observed at each endpoint in the hierarchy.

### Safety Outcomes

RBT-1 was well tolerated, with the primary adverse event (AE) considered related to RBT-1 being photosensitivity, which is a known reaction to the SnPP component of RBT-1. Photosensitivity was dose-dependent, occurring in 6 of 45 patients (13%) treated with low-dose RBT-1 and in 12 of 46 patients (26%) treated with high-dose RBT-1. In general, photosensitivity reactions were transient and mild to moderate in intensity. The median time of onset of photosensitivity was 2.5 days in the low-dose RBT-1 group and 2.0 days in the high-dose RBT-1 group; median time to resolution was 3.5 days in the low-dose RBT-1 group and 7.0 days in the high-dose RBT-1 group. All photosensitivity reactions resolved within 28 days in the LD group and within 93 days in the HD group. Three photosensitivity reactions in the high-dose RBT-1 group resulted in delayed surgery.

### Discussion

This study of RBT-1 met its primary endpoint, demonstrating a statistically significant increase in the levels of cytoprotective proteins (plasma HO-1, IL-10, and ferritin), which are surrogate measures for RBT-1-mediated activation of a preconditioning response. The overall incidence of AKI was low and AKI-related outcomes did not show statistical significance in this unenriched population.

Cardiac surgery especially with cardiopulmonary bypass induces systemic inflammation which can lead to multi-organ dysfunction, impacting clinical outcomes. Importantly, inflammation and oxidative stress exist in a feed-forward loop, magnifying the response of each pathway. The detrimental effect of these cell-damaging mediators can be seen in the phenomenon of “organ crosstalk,” wherein damage in one organ leads to damage in another organ. Just as these harmful mediators can adversely impact distant organs, cytoprotective mediators can also be carried from one organ to another.

The benefits observed with RBT-1 are likely related to mitigation of these adverse effects by activating anti-inflammatory and antioxidant pathways prior to surgery, hence resulting in direct and indirect beneficial effects in various organs. These broad organ protective benefits may result in an improvement in clinical outcomes as manifested by reduced time on ventilator, need for vasopressors, new-onset atrial fibrillation and fluid overload in the short term, and a decrease in cardiopulmonary hospital readmissions in the longer term. To further assess this hypothesis, we explored the effects of RBT-1 in a post hoc analysis using a win ratio based composite endpoint wherein clinical outcomes were assessed in rank order of severity. This assessment day cardiopulmonary readmission, atrial fibrillation, and hospital days suggests clinical improvement in response to RBT-1, which will be confirmed in an upcoming larger study.

The safety profile of RBT-1 showed that it was well tolerated, with the primary drug-related adverse effect being photosensitivity, which was dose-related and time-limited. The SnPP component (a metalloporphyrin) of RBT-1 is likely the cause of photosensitivity as metalloporphyrins are light responsive and may lead to a sunburn in patients exposed to the sun, especially if sun exposure is prolonged or sunscreen is not used.

Given the better safety profile of low-dose RBT-1 (45 mg SnPP/240 mg FeS) with respect to photosensitivity, this dose was selected for the Phase 3 study, wherein the planned primary endpoint will be a hierarchical composite of clinical outcomes in rank order of severity.

### Example 6

152 patients were enrolled at 19 sites across the US, Canada, and Australia (Table 22).

TABLE 22

| | High-dose | Low-dose | Placebo |
|---|---|---|---|
| | (N = 46) | (N = 42) | (N = 44) |
| Age (years), Mean (min, max) | 66.0 ± 11.4 | 64.2 ± 8.6 | 65.7 ± 10.7 |
| Sex | | | |
| Female | 10 (22%) | 11 (26%) | 12 (27%) |
| Male | 36 (78%) | 31 (74%) | 32 (73%) |
| Race | | | |
| White | 41 (89%) | 35 (83%) | 41 (93%) |
| Black | 2 (4%) | 4 (10%) | 2 (5%) |
| Asian | 2 (4%) | 1 (2%) | 1 (2%) |
| American Indian | 1 (2%) | 0 (0%) | 0 (0%) |
| Other | 0 (0%) | 2 (5%) | 0 (0%) |
| Weight (kg), Mean (min, max) | 91.1 ± 19.6 | 97.5 ± 20.9 | 90.3 ± 18.9 |
| BMI (kg/m²), Mean (min, max) | 30.3 ± 6.6 | 32.5 ± 6.3 | 30.0 ± 5.8 |
| EuroSCORE II, Mean (min, max) | 1.7 (1.1-2.7) | 1.2 (0.9-2.7) | 1.5 (0.9-2.3) |
| Low Risk (<3), N (%) | 35 (76%) | 33 (79%) | 36 (84%) |
| Medium Risk (3 to 6), N (%) | 9 (20%) | 4 (10%) | 5 (12%) |
| High Risk (≥6), N (%) | 2 (4%) | 5 (12%) | 2 (5%) |
| Acute Kidney Injury Risk Factors | | | |
| Age ≥ 65 years | 28 (61%) | 24 (57%) | 26 (59%) |
| Diabetes mellitus requiring insulin | 8 (17%) | 7 (17%) | 4 (9%) |
| Congestive heart failure | 7 (15%) | 6 (14%) | 7 (16%) |
| Heart failure (NYHA III/IV) within 1 | 6(13%) | 3 (7%) | 4 (9%) |
| year prior to surgery | | | |
| Previous cardiac surgery with | 1(2%) | 1(2%) | 0(0%) |
| sternotomy | | | |
| Left ventricular ejection fraction ≤ | 5(11%) | 6(14%) | 7(16%) |
| 35% | | | |
| Estimated glomerular filtration rate ≥ | 13(28%) | 13(31%) | 8(18%) |
| 20 to <60 mL/min/1.73 m² | | | |
| Preoperative anemia (hemoglobin < | 0(0%) | 1(2%) | 1(2%) |
| 10 g/dL) | | | |
| Hospitalized for management of | 11(24%) | 6(14%) | 8(18%) |
| cardiac or pulmonary disease | | | |
| Time infusion to surgery, Mean ± SD | 40.9 (26.0-44.7) | 40.9 (28.2-44.2) | 41.3 (24.8-44.8) |
| (hrs) | | | |
| Time on cardiopulmonary bypass, | 1.8(1.3-2.3) | 1.7(1.5-2.4) | 1.6(1.3-2.3) |
| median (IQR), h | | | |
| Surgery Type | | | |
| CABG alone, N (%) | 24(52%) | 23(55%) | 22(50%) |
| Valve alone, N (%) | 11(24%) | 13(31%) | 8(18%) |
| CABG + Valve, N (%) | 11(24%) | 6(14%) | 14(32%) |

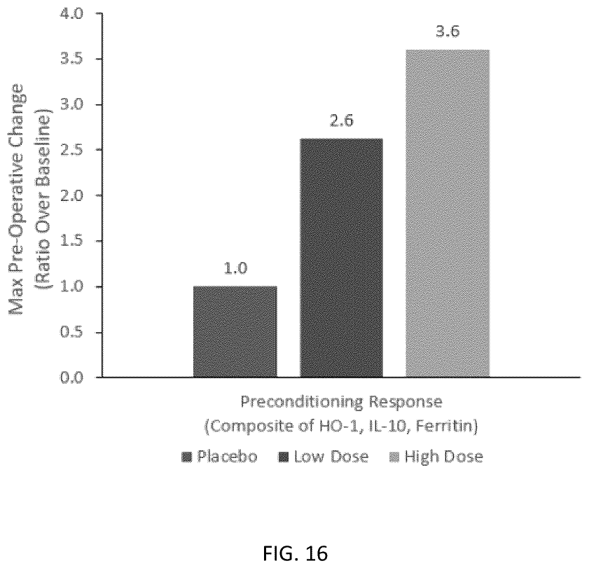

Data are n (%) unless otherwise specified. Percentages are rounded. CABG=coronary artery bypass grafting. EuroSCORE=European system for cardiac operative risk evaluation. NYHA=New York Heart Association. Among the enrolled patients, 135 were randomly assigned to either the high-dose RBT-1 group (n=46), low-dose RBT-1 group (n=45), or placebo group (n=44). FIG. 16 shows the preconditioning response is shown as the geometric least squares mean for the ratio of the maximum pre-operative change over baseline in a composite of heme oxygenase-1 (HO-1), interleukin-10 (IL-10), and ferritin in the intent-to-treat population. HO=heme oxygenase, IL=interleukin.

The planned interim analysis was conducted from May 9 to Jun. 6, 2022 and included the first 62 patients randomized (n=20 high-dose, n=19 low-dose, n=23 placebo). The interim results indicated significant differences in the primary outcome between both high-dose RBT-1 (p<0.0001) and low-dose RBT-1 (p<0.0001) groups compared with the placebo group (Table 23).

TABLE 23

| | | | | High-dose v. placebo | | Low-dose v. placebo | |
|---|---|---|---|---|---|---|---|
| | High- | | | GLSM | | GLSM | |
| | dose | Low-dose | Placebo | Ratio | | Ratio | |
| | n = 46) | (n = 42) | (n = 44) | (95% CI) | P value | (95% CI) | P value |
| Biomarker | 3.60 | 2.63 | 1.00 | 3.58 † | <0.0001 | 2.62 † | <0.0001 |
| response | | | | (2.91- | | (2.11- | |
| GLSM* | | | | 4.31) | | 3.24) | |

Data are GLSM (95% CI) unless otherwise specified.
GLSM = geometric least squares mean.
*GLSM of the ratio of max post-op value over baseline.
† GLSM ratio represents GLSM in the active treatment group over GLSM in the placebo group.

The unblinded statistician recommended the study be continued without enrichment order to provide additional evidence in support of the secondary outcomes and the study sponsor representative agreed. Therefore, an additional 73 patients (approximately 24 per group) were enrolled without enrichment. The safety population consisted of all randomized patients (n=135), all of whom received study treatment. Of those, 132 (98%) patients had biomarker measurements collected (ITT population), for whom the primary outcome was evaluated (n=46 high-dose, n=42 low-dose, n=44 placebo). From the ITT population, 121 patients had surgery on-time (24 to 48 hours after infusion) and constituted the MITT population, for whom secondary and clinical outcomes were evaluated (n=41 high-dose, n=39 low-dose, n=41 placebo).

At randomization, baseline characteristics were generally similar among intervention groups in the safety population (Supplementary Table S3), ITT population (Table 18), and MITT population (Supplementary Table S4). In the ITT population, the EuroSCORE and AKI risk factors were often numerically lower in the placebo group versus both RBT-1 groups. However, the contrary was observed for the incidence of combined surgery (CABG+Valve). The time between infusion and start of surgery, as well as time on cardiopulmonary bypass, were similar between the 3 treatment groups.

The primary composite outcome in the ITT population was 3.60 in the high-dose RBT-1 group, 2.63 in the low-dose RBT-1 group, and 1.00 in the placebo group (Table 24, FIG. 16). Both high-dose and low-dose RBT-1 were associated with an increased preconditioning biomarker response compared with placebo (high-dose vs. placebo; GLSM ratio, 3.58; 95% Cl, 2.91-4.41; p<0.0001; and low-dose vs. placebo; GLSM ratio, 2.62; 95% Cl, 2·11-3·24; p<0.0001).

Secondary outcomes of AKI incidence and sustained reduction in urine output were numerically lower with both doses of RBT-1 compared with placebo but the differences were not significant (Table 24).

TABLE 24

| | High- | Low- | | High-dose v. | Low-dose v. |
|---|---|---|---|---|---|
| | dose | dose | Placebo | placebo, risk ratio | placebo, risk ratio |
| | (n = 41) | (n = 39) | (n = 41) | (95% CI) | (95% CI) |
| Acute kidney injury | 7(17%) | 7(18%) | 8(20%) | 0.88(0.35-2.19) | 0.92(0.37-2.30) |
| Sustained reduction in | 2(5%) | 2(5%) | 4(10%) | 0.5(0.10-2.58) | 0.53(0.10-2.71) |
| urine output | | | | | |
| Tubular injury | 7.9 | 10.8 | 6.1 | 1.29 (0.71-2.35) | 1.78 (0.96-3.30) |
| biomarker | | | | | |
| response, GLSM* | | | | | |
| Biomarker response, | 3.60 | 2.62 | 1.00 | 3.61 (2.91-4.47) | 2.62 (2.11-3.27) |
| GLSM* | | | | | |

Data are n (%) unless otherwise specified.
Percentages are rounded.
GLSM = geometric least squares mean.
*GLSM of the ratio of max post-op value over baseline.
^{†}GLSM ratio represents GLSM in the active treatment group over GLSM in the placebo group.

The composite of renal tubular injury biomarkers occurred with similar frequency in the RBT-1 and placebo groups, and these biomarkers did not correlate with the maximum change in serum creatinine (data not shown). The primary composite outcome results in the MITT population were consistent with the results in the ITT population.

RBT-1 was generally well tolerated by patients. The primary drug-related AE was photosensitivity, a known reaction to the SnPP component of RBT-1 (Table 25).

TABLE 25

| | High- | Low- | | High-dose v. | Low-dose v. |
|---|---|---|---|---|---|
| | dose | dose | Placebo | placebo, risk ratio | placebo, risk ratio |
| | (n = 41) | (n = 39) | (n = 41) | (95% CI) | (95% CI) |
| Primary drug- | | | | | |
| related adverse | | | | | |
| event | | | | | |
| Photosensitivity | 12 (26%) | 6 (13%) | 0 (0%) | 26.1(14.2 to 41.1) | 13.3(3.8 to 27.0) |
| Reaction | | | | | |
| Deaths | | | | | |
| Death | 2 (4%) | 1 (2%) | 3 (7%) | −2.5(−15.1 to 8.9) | −4.6(−16.5 to 5.8) |
| Adverse Events | | | | | |
| Subjects with at | 44 (96%) | 40 (89%) | 40 (91%) | 4.7(−6.9 to 17.8) | −2.0(−16.2 to 12.0) |
| least one adverse | | | | | |
| event | | | | | |

Photosensitivity was dose-dependent, occurring in 12 of 46 patients (26%) treated with high-dose RBT-1 and in 6 of 45 patients (13%) treated with low-dose RBT-1 in the safety population. In general, in intensity. The median time of onset of photosensitivity was 2.0 days in the high-dose RBT-1 group and 2.5 days in the low dose RBT-1 group; median time to resolution was 7.0 days in the high-dose RBT-1 group and 3.5 days in the low-dose RBT-1 group. All photosensitivity reactions resolved within 93 days in the high-dose RBT-1 group and within 28 days in the low-dose RBT-1 group. Three photosensitivity reactions in the high-dose RBT-1 group resulted in delayed surgery. Other AEs of general interest are also provided in Table 25.

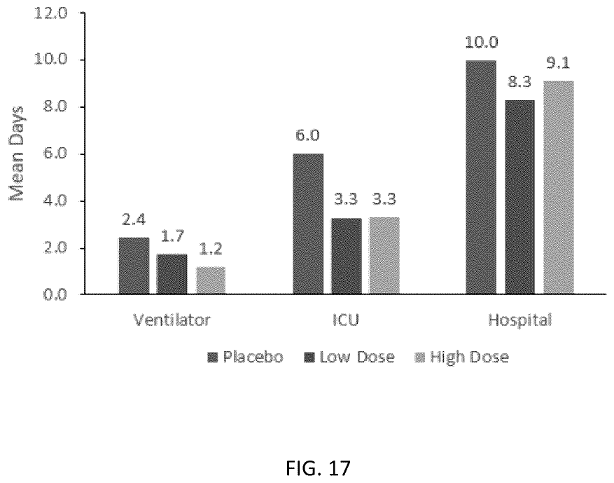

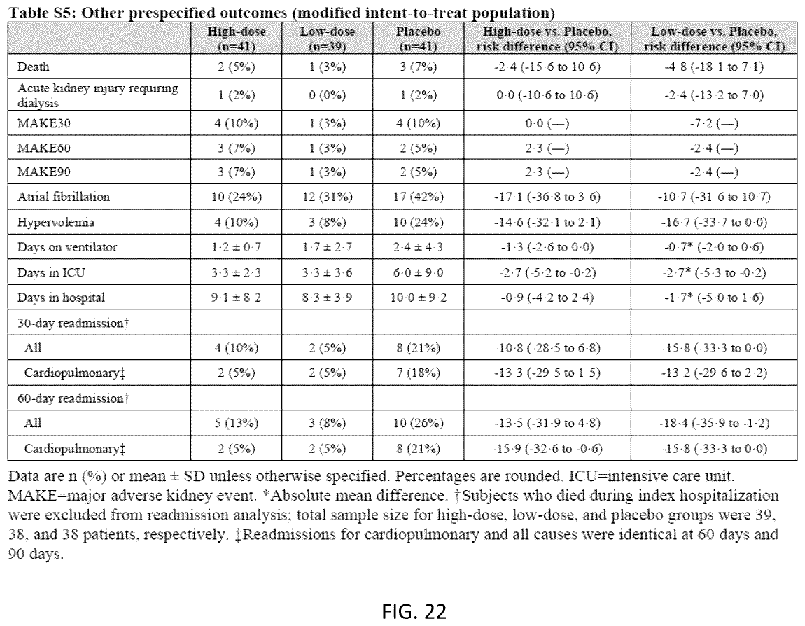

Post-operative complications included 2 (5%) deaths in the high-dose RBT-1 group, 1 (3%) death in the low-dose RBT-1 group, and 3 (7%) deaths in the placebo group FIG. 22 (Table S5). Dialysis for AKI was needed in 1 (2%) patient in the placebo group but none in either RBT-1 group. The number of patients with MAKE was relatively low, and no statistical differences between groups were observed. Post-operative atrial fibrillation and hypervolemia were numerically lower in both RBT-1 groups compared with placebo, but the differences were not significant. The mean time on ventilator was 1.2 days, 1.7 days, and 2.4 days in the high-dose RBT-1, low-dose RBT-1, and placebo groups, respectively. FIG. 17 and FIG. 22 (Table S5). The mean time in ICU was 3.3 days in both RBT-1 groups and 6.0 days in the placebo group. The mean time in hospital was 9.1 days, 8.3 days, and 10.0 days in the high-dose RBT-1, low-dose RBT-1, and placebo groups, respectively. Finally, 2 (5%) patients each in both RBT-1 groups were readmitted to the hospital at 30-days post-discharge for a cardiopulmonary diagnosis compared with 7 patients (18%) in the placebo group. Through 60 days and 90 days post-discharge, the cardiopulmonary readmission rate remained the same in both RBT-1 groups with 2 (5%) patients each readmitted compared with an increase to 8 (21%) patients in the placebo group. All-cause readmissions showed similar results.

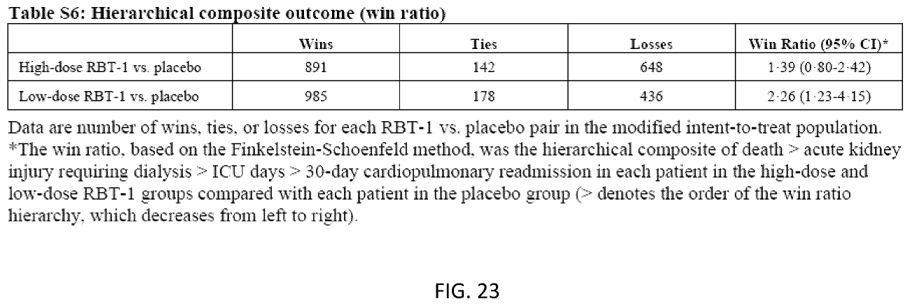

Given the suggested multi-organ benefit of RBT-1, we explored the effects of RBT-1 in a post hoc composite analysis (win ratio) wherein clinical outcomes were assessed in rank order of severity (death>AKI requiring dialysis>ICU days>30-day cardiopulmonary readmission) in the MITT population. The win ratio was 1.39 (95% Cl, 0.80-2.42) in patients treated with high dose RBT-1 and 2.26 (95% Cl, 1.23-4.15) with low-dose RBT-1 compared with placebo. FIG. 23 (Table S6).

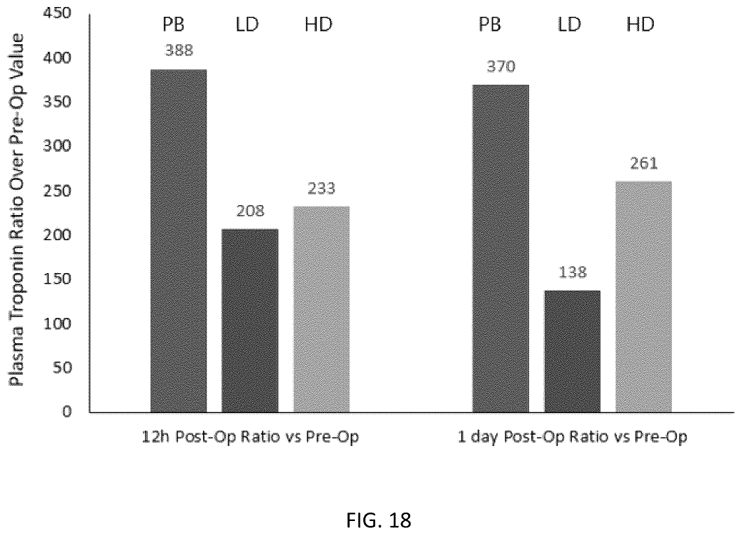

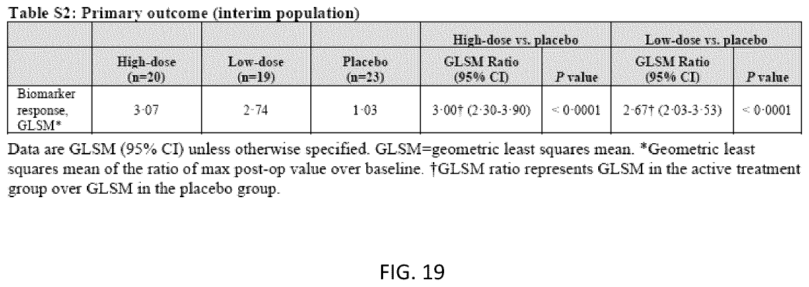

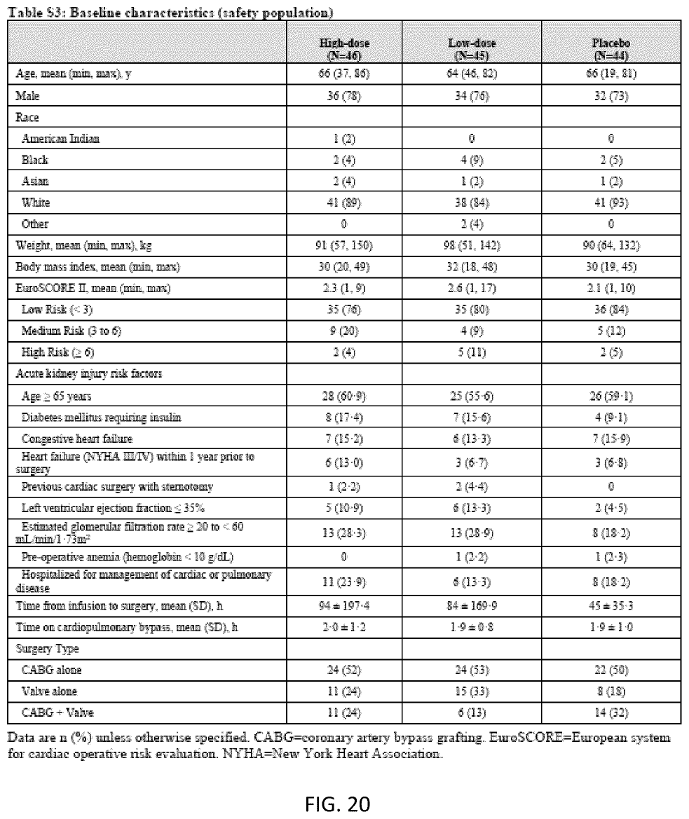

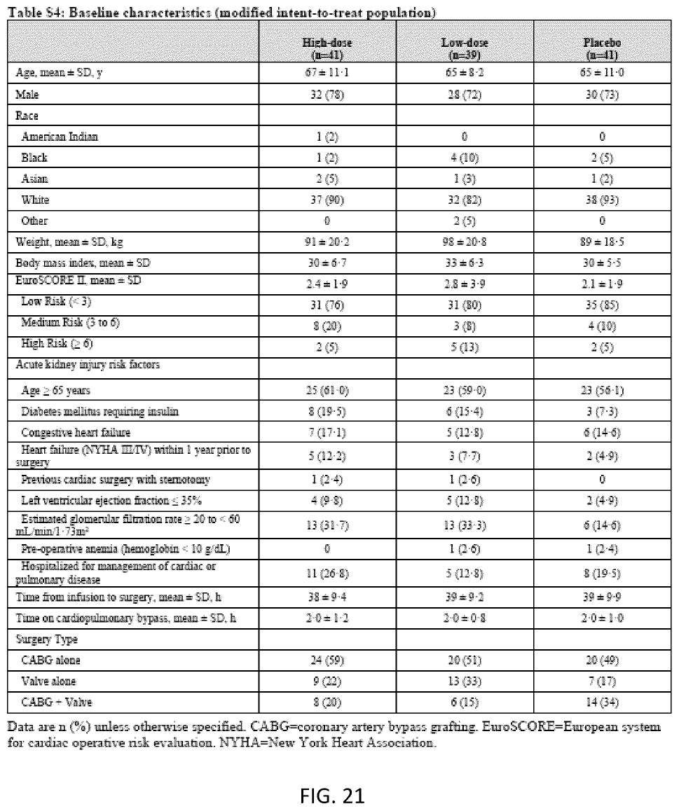

In an analysis of myocardial injury using troponin I, the rise in troponin I after cardiac surgery (GLSM of the ratio between 1-day post-operative values and preoperative baseline values) was numerically lower in the high-dose RBT-1 group compared with placebo but the difference was not significant (GLSM ratio, 0.71; 95% Cl, 0.32-1.58). However, the rise in postoperative troponin I was reduced in the low-dose RBT-1 group compared with placebo (GLSM ratio, 0.37; 95% Cl, 0.17-0.82) FIG. 18 shows the increase in plasma troponin I from pre-operative baseline to 12 hours (left series) and 1 day (right series) after cardiac surgery. The analysis population, derived from the modified intent-to treat population, excluded patients who had undergone mitral valve repair or replacement, ablation, or septal myectomy due to the expected significant increase in troponin I levels following these major surgeries. The analysis population, derived from the MITT population, excluded patients who had undergone mitral valve repair or replacement, ablation, or septal myectomy due to the expected large increase in troponin I levels following these major surgeries.

### Discussion

This study of RBT-1 met its primary outcome, demonstrating a statistically significant increase in the levels of cytoprotective proteins (plasma HO-1, IL-10, and ferritin), which are surrogate measures for RBT-1-mediated activation of a preconditioning response. The RBT-1 biomarker response was consistent with the Phase 1b study. The overall incidence of AKI was low and differences in AKI-related outcomes were not significant 430 in this population. Despite the relatively small sample size of this study, we observed a reduction in adverse post-operative outcomes, suggesting that RBT-1 may improve recovery after cardiac surgery.

Cardiac surgery, especially with cardiopulmonary bypass, induces systemic inflammation, which can lead to multi-organ dysfunction, impacting clinical outcomes. Importantly, inflammation and oxidative stress exist in a feed-forward loop, magnifying the response of each pathway. The detrimental effect of these cell-damaging mediators can be seen in the phenomenon of “organ crosstalk,” wherein damage in one organ leads to damage in another.

The benefits observed with RBT-1 are likely related to mitigation of these adverse effects by activating anti-inflammatory and antioxidant pathways prior to surgery, thereby resulting in direct and indirect beneficial effects in various organs. For example, the RBT-1-mediated anti inflammatory response may prevent extravasation of fluid into tissues due to capillary leakage, thus reducing the need for fluid (volume) replacement and ultimately fluid overload as observed by the reduction in hypervolemia in RBT-1-treated patients. The broad organ protective benefits of RBT-1 may result in an improvement in clinical outcomes as manifested by reduced time on ventilator, time in ICU, need for vasopressors, new-onset atrial fibrillation, and fluid overload in the short-term and a decrease in cardiopulmonary hospital readmissions in the long-term. To further assess this hypothesis, we explored the effects of RBT-1 in a post hoc analysis using a win ratio based on a composite of clinical outcomes assessed in rank order of severity. This assessment, which consisted of death, AKI requiring dialysis, ICU days, and 30-day cardiopulmonary readmission, suggested clinical improvement in response to RBT-1, which will be confirmed in an upcoming Phase 3 study.

The safety profile of RBT-1 showed that it was well tolerated, with the primary drug related AE being photosensitivity, which was dose-related and time-limited. The SnPP component (a metalloporphyrin) of RBT-1 is likely the cause of photosensitivity as metalloporphyrins are light responsive and may lead to a sunburn in patients exposed to the sun, especially if sun exposure is prolonged or sunscreen is not used. The low-dose of RBT-1 (45 mg SnPP/240 mg FeS) is planned for the definitive Phase 3 trial due to comparable efficacy and fewer photosensitivity reactions compared with high-dose RBT-1.

As a Phase 2 trial, the aim was to investigate whether RBT-1 administered before surgery would elicit a preconditioning response in patients undergoing CABG and/or heart valve surgery and was not powered (i.e., relatively small sample size) to demonstrate statistically significant reductions in clinical outcomes; a larger Phase 3 trial is needed to demonstrate such effects. This study was conducted during the COVID-19 pandemic, which may have impacted the LOS in ICU; however, to minimize variability in standard of care, patients were randomized at the site level. Although composite outcomes showing statistically significant improvement were evaluated post hoc, a consistent trend of improvement with RBT-1 treatment was observed. One (0.7%) patient had incomplete serum creatinine values required for the MAKE outcome at Day 60 and 90 due to COVID-19 that prevented the patient from returning to the hospital for lab collection. However, we imputed laboratory values for this patient. Given the exploratory nature of the trial, the type I error rate was not controlled using bias adjustment methods.

This study of RBT-1 met its primary outcome, demonstrating a statistically significant increase in the levels of cytoprotective proteins (plasma HO-1, IL-10, and ferritin), which are surrogate measures for RBT-1-mediated activation of a preconditioning response. Given the positive trends in clinical outcomes and adequate safety profile, a Phase 3 study of RBT-1 is planned, wherein the primary outcome will be a hierarchical composite of clinical outcomes.

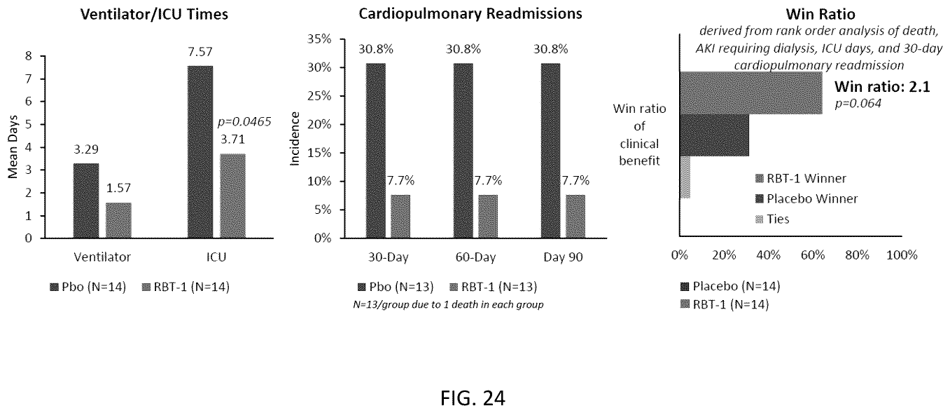

Patients undergoing CABG/cardiac value surgery showed an improvement in clinical outcomes, including (a) ventilator time, (b) ICU time, and (c) cardiopulmonary readmission as shown in FIG. 24. Specifically, the ventilator time was reduced from 3.29 for the placebo group to 1.57 for the RBT-1 treatment group. The ICU time was reduced from 7.57 for the placebo group to 3.71 for the RBT-1 treatment group. The 30-, 60-, and 90-day cardiopulmonary readmissions were reduced from 30.8% for the placebo group to 7.7% for the RBT-1 treatment group. These results are further demonstrated by a win ratio of 2.1 for RBT-1 versus placebo as shown in FIG. 24.

Additional observations from the study include the following:

A significant reduction of 49% in the incidence of post-operative anemia, as reported by the investigators, was observed in response to RBT-1 compared with placebo (p=0.0447).

A 33% reduction in the need for blood transfusion was observed in the RBT-1 group compared with placebo.

Similarly, a 42% reduction in the need for iron supplementation was observed in the RBT-1 group compared with placebo.

When comparing all patients who required both blood transfusion and iron, a statistically significant reduction was observed in the RBT-1 group compared with placebo (RBT-1:0.0% vs Placebo: 12.2%; p=0.0046).

Importantly, hemoglobin levels at discharge in patients who received RBT-1 were comparable to those who received placebo despite lesser rates of blood transfusion and iron supplementation (RBT-1:9.8 g/dL vs Placebo: 9.8 g/dL).

### Example 7

The SnPP produced according to Example 1 is administered with iron sucrose bicarb by intravenous infusion from a bolus between 24 and 48 hours before a scheduled CABG surgery. The dose is 45 mg SnPP and 240 mg iron sucrose bicarb.

### Example 8

The SnPP produced according to Example 1 is administered with iron sucrose bicarb by intravenous infusion from a bolus between 24 and 48 hours before a scheduled CABG surgery. The dose is 45 mg SnPP and 240 mg iron sucrose bicarb.

### Example 9

A patient who has been found to be photosensitive (skin is susceptible to sunburn) is treated by intravenous infusion of RBT-1 between 24 and 48 hours before a scheduled CABG surgery. The RBT-1 dose is 45 mg stannic protoporfin composition according to Example 1 and 240 mg iron sucrose. After infusion of RBT-1 and before any exposure to sunlight, the patent's exposed skin is treated with SPF 50 sunscreen. The patent undergoes CABG surgery. After surgery, SPF 50 sunscreen is administered prior to any sunlight exposure within six days from the surgery.

### Example 10

A patient who has been found to be photosensitive (skin is susceptible to sunburn) is treated by intravenous infusion of RBT-1 between 24 and 48 hours before a scheduled CABG surgery. The RBT-1 dose is 45 mg stannic protoporfin composition according to Example 1 and 240 mg iron sucrose. Exposure to sunlight is avoided from the time of RBT-1 administration and the surgery. The patent undergoes CABG surgery. After surgery, SPF 50 sunscreen is administered prior to any sunlight exposure within six days from the surgery.

### Example 11

A patient who is susceptible to hospital readmission for cardiopulmonary purposes after CABG surgery is treated by is treated by intravenous infusion of RBT-1 between 24 and 48 hours before a scheduled CABG surgery. The RBT-1 dose is 45 mg stannic protoporfin composition according to Example 1 and 240 mg iron sucrose. The patent undergoes CABG surgery. After surgery, the patient is at least 60% less likely to need readmission to the hospital for cardiopulmonary purposes.

### Example 12

A patient who is susceptible post-operative complications after CABG surgery is treated by is treated by intravenous infusion of RBT-1 between 24 and 48 hours before a scheduled CABG surgery. The RBT-1 dose is 45 mg stannic protoporfin composition according to Example 1 and 240 mg iron sucrose. The patent undergoes CABG surgery. After surgery, the patient is less likely to suffer from the following complications (a) greater than three days in the intensive care unit, (b) greater than 24 hours on a ventilator, (c) readmission for cardiopulmonary surgery, (d) need for a blood transfusion, (e) new onset post-operative atrial fibrillation (POAF) during hospitalization, or a combination of two or more of (a)-(e).

### Example 13

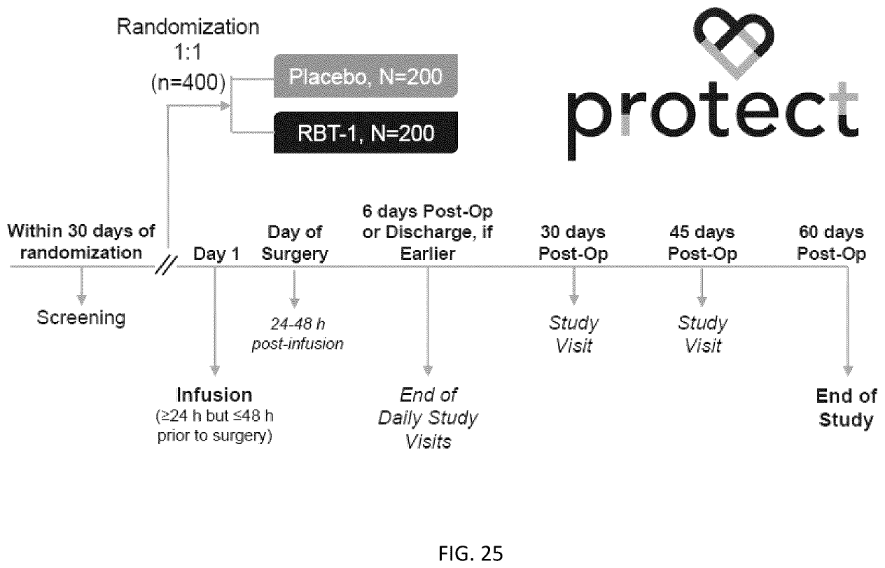

A phase III study is conducted for evaluating reducing the risk of postoperative complications in patients undergoing cardiothoracic surgery. FIG. 25 shows the design of this study. The primary objective of the study will be to evaluate the efficacy of RBT-1 compared with placebo on a hierarchical composite (win ratio) of: death, incidence of acute kidney injury (AKI) requiring dialysis, intensive care unit (ICU) days, and 30-day cardiopulmonary readmission rates. The secondary objective of the study will focus on post-operative complications, ICU days, 30-day cardiopulmonary readmission rates, and safety. The post-operative complications that will be studied include death, AKI requiring dialysis, >3 days in ICU, >24 hours on a ventilator, 30-day cardiopulmonary readmission, need for blood transfusion during index hospitalization, and new-onset post-operative atrial fibrillation (POAF) during index hospitalization.

### Example 14

A demetallation of hemin to produce protoporphyrin IX was made according to an embodiment of invention, as follows:

A 1 L round-bottom flask was fitted with a mechanical stirrer, reflux condenser, thermocouple, and a heating mantle with controller. About 25 grams of hemin were added in addition to about 500 ml of formic acid. Stirring commenced and about 78 mL of cyclohexene was added while the flask was heated to about 70° C.

About 5.36 grams of iron powder were added in 5×1.08 g portions every 5 minutes. The iron addition was very exothermic and the inventors observed frothing with each addition. A 51.5% conversion was observed after about 0.5 hour, 56% conversion was achieved after about 1 hour, 74% conversion was achieved after about 2 hours, and 88% conversion was achieved after about 3.5 hours.

After about 4 hours, the flask was cooled to about 50° C. and the mixture was filtered through a fritted glass filter filled with celite, and washed with about 167 mL of formic acid. The resulting solution was added to an ammonium acetate solution (with stirring), which was prepared by adding about 249 grams of NH₄OAc to about 1826 ml water in a 4 L Erlenmeyer flask. The resulting mixture was stirred for about 0.5 hour at room temperature and filtered through 3-fast flow filter papers on a buchner funnel. The product was then washed with 5×50 mL water, air dried, and then dried on a lyophilizer overnight. The yield was about 15.09 grams (70%-HPLC purity: 97.13%, mesoporphyrin: 0.5%).

### Example 15

Stannous chloride is dissolved in pyridine under inert atmosphere, glacial acetic acid is added and the mixture is heated at 50° C. Protoporphyrin IX from EXAMPLE 14 is then added and stirred and heated for a minimum of 24 hours and monitored for completion by HPLC. The reaction is cooled to room temperature and filtered. The product is then triturated first with water, then 2 M HCl(aq) and then again with water. The product is then dried to remove residual solvents to yield stannic protoporfin.

### Example 16

A demetallation of hemin to produce protoporphyrin IX was made according to an embodiment of invention, as follows:

A 1 L round-bottom flask was fitted with a mechanical stirrer, reflux condenser, thermocouple, and a heating mantle with controller. About 25 grams of hemin were added in addition to about 0.25 grams of benzyl triethylammonium chloride and 500 mL of formic acid. Stirring commenced and about 39 mL of cyclohexene was added while the flask was heated to about 60° C.

About 5.36 grams of iron powder were added in 5×1.08 g portions every 5 minutes. The mixture was stirred overnight at about 60° C., then cooled to about 50° C., filtered through a fritted glass filter filled with celite, and washed with about 175 ml of formic acid. The resulting solution was added to an ammonium acetate solution (with stirring), which was prepared by adding about 249 grams of NH4OAc to about 1826 mL water in a 4 L Erlenmeyer flask. The resulting mixture was stirred for about 0.5 hour at room temperature and filtered through 3-fast flow filter papers on a buchner funnel. The product was then washed with 5×50 ml water, air dried, and then dried on a lyophilizer overnight. The yield was about 21.37 grams (99%-HPLC purity: 89%).

### Example 17

Stannous chloride is dissolved in pyridine under inert atmosphere, glacial acetic acid is added and the mixture is heated at 50° C. Protoporphyrin IX from EXAMPLE 16 is then added and stirred and heated for a minimum of 24 hours and monitored for completion by HPLC. The reaction is cooled to room temperature and filtered. The product is then triturated first with water, then 2 M HCl(aq) and then again with water. The product is then dried to remove residual solvents to yield stannic protoporfin.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

1. A stannic protoporfin composition comprising a compound of Formula (I) 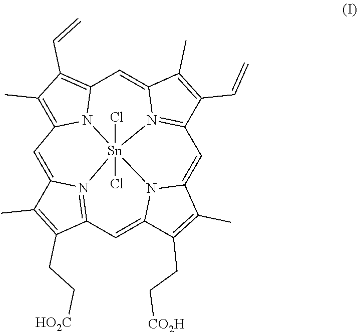
wherein the composition comprises a level of mesoporphyrin of Formula (III) 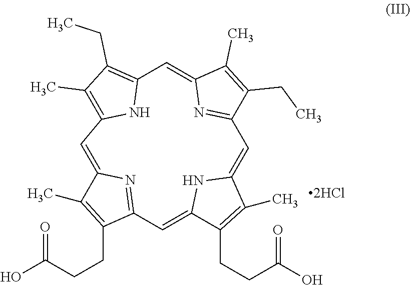 [SMILES: CCC1=C(C)c2cc3[nH]c(cc4nc(cc5[nH]c(cc1n2)c(C)c5CCC(=O)O)C(CCC(=O)O)=C4C)c(C)c3CC]
that is less than 1.0 wt. % and a total impurity level below 3 wt. %.

2. The stannic protoporfin composition of claim 1, wherein the total impurity level is less than 1.5%.

3. The stannic protoporfin of claim 1, wherein the level of mesoporphyrin is in the range of 0.01 to 0.9% by weight, 0.1 to 0.9% by weight, 0.2 to 0.8% by weight, 0.3 to 0.7% by weight, 0.4 to 0.6% by weight, or about 0.5% by weight

4. The stannic protoporfin of claim 1, wherein the total level of impurities includes mesoporphyrin and degradation products of tin protoporphyrin and intermediates of tin protoporphyrin.

5. The stannic protoporfin of claim 1, wherein the total level of impurities is in the range of 0.01 to 1.5% by weight, 0.01 to 0.9% by weight, 0.1 to 0.9% by weight, 0.2 to 0.8% by weight, 0.3 to 0.7% by weight, 0.4 to 0.6% by weight, or about 0.5% by weight.

6. The stannic protoporfin of claim 1, wherein the total level of impurities is determined by gas chromatography or HPLC.

7. The stannic protoporfin of claim 1, wherein the level of mesoporphyrin is determined by gas chromatography or HPLC.

8. A method of making Stannic protoporfin comprising the steps of:
(a) dissolving hemin in hot formic acid to form a first intermediate composition;
(b) adding iron powder to the first intermediate composition to form a second intermediate composition;
(c) adding a filtrate of the second intermediate composition to an aqueous solution of NH₄OAc to precipitate a third intermediate composition;
(d) dissolving the third intermediate composition in pyridine at an elevated temperature to form a fourth intermediate composition;
(e) filtering the fourth intermediate composition to form a filtrate; and
(f) precipitating a first intermediate compound according to Formula II:

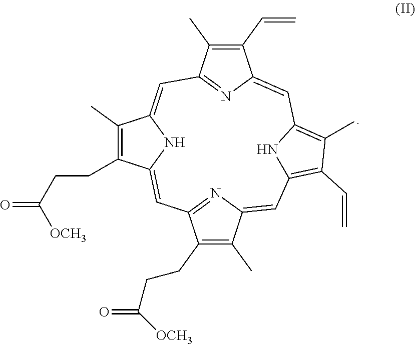 [SMILES: C=CC1=C(C)c2cc3[nH]c(cc4nc(cc5[nH]c(cc1n2)c(C)c5C=C)C(C)=C4CCC(=O)OC)c(CCC(=O)OC)c3C]

9. The method of claim 8, further comprising adding the compound of Formula (II) to a mixture of stannous chloride, pyridine and glacial acetic acid to make a stannic protoporfin according to Formula I:

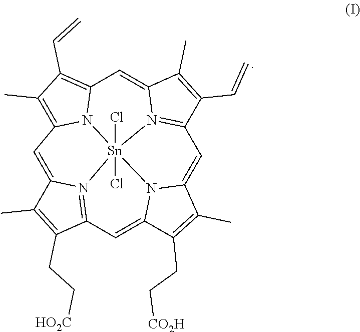

10. The method of claim 8, wherein adding the compound of Formula (II) to a mixture of stannous chloride, pyridine and glacial acetic acid is conducted in an inert environment.

11. The method of claim 8, wherein step (a) further comprises the addition of an olefin.

12. The method of claim 11, wherein step (a) further comprises the addition of a phase transfer catalyst.

13. A stannic protoporfin composition made according to the process of claim 8.

14. A pharmaceutical composition comprising the stannic protoporfin composition of claim 1, and an iron pharmaceutical composition comprising: iron sucrose; bicarbonate; and a pharmaceutically acceptable aqueous carrier

15. The pharmaceutical composition of claim 14, wherein the iron sucrose is present in pharmaceutically effective amount, the iron being present in both iron (II) and iron (III) form, and the iron sucrose has a MW according to GPC of between 33,000 and 38,000 Daltons.

16. A method of reducing post operative complications of a human patient from injury based on a scheduled or anticipated insult comprising administering to the patient the composition of claim 14.

17. A method for demetallation of a vinyl group-containing metallomacrocycle, comprising:
reacting the vinyl group-containing metallomacrocycle with a transition metal having an oxidation number of 0 in a mixture comprising an acid and an olefin.

18-41. (canceled)

42. A composition, comprising:
a vinyl group-containing metallomacrocycle,
an acid,
an olefin, and
a transition metal having an oxidation number of 0.

43-45. (canceled)

46. A method of reducing post operative complications of a human patient from injury based on a scheduled or anticipated surgical operation, the method comprising administering to the human patient a composition comprising a therapeutically effective amount of (i) an iron compound; and (ii) stannic protoporfin in a dose of 20-80 mg before the surgical operation, wherein the human patient is susceptible to photodermatoses.

47-78. (canceled)

79. A method of reducing post operative complications of a human patient from injury based on a scheduled or anticipated insult to an organ of the human patient, the method comprising:
(a) administering to the patient a therapeutically effective composition comprising (i) an iron compound; and (ii) stannic protoporfin, and
(b) applying sunscreen to the human patient's skin around the time of administering to the human patient the therapeutically effective composition or within six days after the scheduled or anticipated surgical operation,
wherein the therapeutically effective composition is administered before the scheduled or anticipated insult to the organ occurs.

80-112. (canceled)

113. A method of reducing hospital readmission for cardiopulmonary purposes of a human patient after a surgical operation by at least 60% comprising administering to the patient a therapeutically effective composition comprising an amount of (i) an iron compound; and (ii) stannic protoporfin before the surgical operation.

114-115. (canceled)

116. A method of reducing post operative complications of a human patient from injury based on a scheduled or anticipated surgical operation comprising administering to the patient a therapeutically effective composition comprising an amount of (i) an iron compound; and (ii) stannic protoporfin before the surgical operation, wherein the post operative complications include: (a) greater than three days in the intensive care unit, (b) greater than 24 hours on a ventilator, (c) readmission for cardiopulmonary surgery, (d) need for a blood transfusion, (e) new onset post-operative atrial fibrillation (POAF) during hospitalization, or a combination of two or more of (a)-(e).

117-150. (canceled)
